(19) United States
(12) Patent Application Publication
(10) Pub. No.: US 2024/0221922 A1
(43) Pub. Date: Jul. 4, 2024

(54) CHAOTIC STORAGE OF PRESCRIPTION MEDICATIONS

(71) Applicant: SUPPORTING INITIATIVES TO REDISTRIBUTE UNUSED MEDICINE (SIRUM), Palo Alto, CA (US)

(72) Inventors: Adam S. KIRCHER, Tallahassee, FL (US); Kiah J. WILLIAMS, Mountain View, CA (US); George J. WANG, Menlo Park, CA (US)

(73) Assignee: SUPPORTING INITIATIVES TO REDISTRIBUTE UNUSED MEDICINE (SIRUM), Palo Alto, CA (US)

(21) Appl. No.: 18/459,794

(22) Filed: Sep. 1, 2023

Related U.S. Application Data
(63) Continuation of application No. 16/133,603, filed on Sep. 17, 2018, now abandoned.

(51) Int. Cl. G16H 40/20; G06K 19/06; G06K 19/07

(52) U.S. Cl. CPC G16H 40/20; G06K 19/06028; G06K 19/0723

(57) ABSTRACT

Provided herein, inter alia, are methods for storing, dispensing, and/or dispositioning pharmaceutical medication(s). These methods use, e.g., location identifiers that identify the location of pharmaceutical medications in an inventory, which allows the storage of medications in the inventory using non-alphabetical ordering, as is the typical method for organizing a pharmacy inventory. This provides, e.g., a more flexible and efficient pharmaceutical inventory that can be accessed with improved safety and reduced opportunities to introduce errors in the dispensing and storage of pharmaceutical medications.

### CROSS-REFERENCE TO RELATED APPLICATIONS

This application is a continuation of U.S. application Ser. No. 16/133,603, filed Sep. 17, 2018, which is incorporated herein by reference in its entirety.

### FIELD

The present disclosure relates generally to the storage, dispensing, and/or dispositioning of pharmaceutical medications, e.g., from a pharmacy inventory.

### BACKGROUND

Pharmacy inventories are constantly in flux, due to factors such as medication expiry, recalls, discontinued drugs, inventory management (e.g., reducing excess inventory), and use of generic or therapeutic equivalents, in addition to the constant streams of incoming inventory and outgoing prescriptions. Traditionally, pharmaceutical medications in a pharmacy inventory are arranged in alphabetical order, either with every medication being ordered alphabetically on the shelves, or with medications generally classified by route of administration and/or form of preparation and then organized alphabetically within each category (see, e.g., www.who.int/management/resources/drugs/practicalpharmacy21b.pdf). However, this method of inventory leads to disadvantages. For example, alphabetical ordering means that receiving a new drug starting with an earlier letter requires shifting most of the inventory to accommodate storing the drug, e.g., at the beginning of a shelf or section. Alphabetical ordering also leads to inefficiently storing drugs at low densities, e.g., to reserve a space for a drug not currently in stock, since it occupies the same position in the inventory. Finally, alphabetical ordering can lead to errors such as dispensing the wrong medication or storing the wrong medication at a particular inventory location due to similar names, or drugs with the same name but different strength or dosage form, stored adjacent to one another.

It is also highly difficult to maintain a live pharmacy inventory. Using existing methods, this would require extensive and frequent efforts to go through the inventory shelves and count individual medications. Requiring pharmacists or pharmacy technicians to update inventory each time medications are stored, dispensed, or dispositioned from stock inventory would impose a huge burden in time and labor.

Therefore, a need exists for improved methods for storing, dispensing, and dispositioning pharmaceutical medications. In particular, methods that allow for a live inventory and reduce the potential for medication errors are highly advantageous.

### BRIEF SUMMARY

To meet these and other demands, the present disclosure provides a methods for storing, dispensing, and/or dispositioning pharmaceutical medication(s) in an inventory. The use of location identifiers (e.g., based on one or more criteria, discussed infra) allows for a flexible, live inventory partially or fully independent of alphabetical ordering, thus providing a more robust, error-free, and efficient inventory, e.g., than one based primarily on alphabetical ordering by drug name.

Certain aspects of the present disclosure relate to methods for storing a pharmaceutical medication in an inventory, comprising: (a) receiving a pharmaceutical medication; (b) at an electronic device with a processor and memory, inputting data related to the pharmaceutical medication, wherein the data identify: (1) name and quantity of at least a portion of the pharmaceutical medication, and (2) a location for at least a portion of the pharmaceutical medication in an inventory, wherein the ordering of pharmaceutical medications in the inventory is not alphabetical by pharmaceutical medication name; (c) creating a record associated with at least a portion of the pharmaceutical medication in the memory of the electronic device, wherein the record identifies the name and quantity of the portion of the pharmaceutical medication and the location for the portion of the pharmaceutical medication in the inventory; and (d) storing the portion of the pharmaceutical medication at the location in the inventory.

Other aspects of the present disclosure relate to methods for dispensing one or more pharmaceutical medications, comprising: (a) receiving an order for one or more pharmaceutical medications, wherein the order indicates a name and a quantity for each of the one or more pharmaceutical medications; (b) at an electronic device with a processor and memory, inputting data related to at least a portion of the one or more pharmaceutical medications, wherein the data identify the name and quantity for the portion of the one or more pharmaceutical medications; (c) receiving a location identifier for at least a portion of the one or more pharmaceutical medications from the electronic device, wherein the location identifier identifies a location of the portion of the one or more pharmaceutical medications in an inventory, and wherein the ordering of pharmaceutical medications in the inventory is not alphabetical by pharmaceutical medication name; (d) retrieving at least a portion of the one or more pharmaceutical medications from the inventory based on the location identifier(s); and (e) dispensing at least a portion of the one or more pharmaceutical medications retrieved in (d) based on the received order.

Other aspects of the present disclosure relate to methods for dispositioning one or more pharmaceutical medications, comprising: (a) at an electronic device with a processor and memory, inputting data identifying one or more of: an expiration date for one or more pharmaceutical medications and a name for one or more pharmaceutical medications (including, e.g., a medication identification code such as NDC); (b) receiving from the electronic device a location identifier for at least a portion of the one or more pharmaceutical medications, wherein the location identifier identifies a location of at least a portion of the one or more pharmaceutical medications in an inventory, and wherein the ordering of pharmaceutical medications in the inventory is not alphabetical by pharmaceutical medication name; (c) retrieving at least a portion of the one or more pharmaceutical medications from the inventory based on the location identifier; and (d) dispositioning at least a portion of the one or more pharmaceutical medications retrieved in (c).

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 provides a flowchart showing exemplary methods for storing pharmaceutical medication(s), in accordance with some embodiments.

FIG. 2 provides a flowchart showing exemplary methods for dispensing or dispositioning pharmaceutical medication(s), in accordance with some embodiments.

FIG. 3 provides a flowchart showing exemplary methods for storing, dispensing, and dispositioning pharmaceutical medication(s), in accordance with some embodiments.

FIG. 4 shows an exemplary storage bin, in accordance with some embodiments.

FIG. 5 shows exemplary inventory shelves and bins, in accordance with some embodiments.

FIG. 6 illustrates a typical computing system that may be employed to implement some or all processing functionality in certain implementations.

FIG. 7 illustrates an exemplary environment for some systems and processes for inputting data and/or receiving information related to medication storage, dispensing, and/or dispositioning, in accordance with some implementations.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Certain aspects of the present disclosure relate to methods for storing a pharmaceutical medication in an inventory. In some embodiments, the methods comprise (a) receiving a pharmaceutical medication; (b) at an electronic device with a processor and memory, inputting data related to the pharmaceutical medication, wherein the data identify: (1) name and quantity of at least a portion of the pharmaceutical medication, and (2) a location for at least a portion of the pharmaceutical medication in an inventory; (c) creating a record associated with at least a portion of the pharmaceutical medication in the memory of the electronic device, wherein the record identifies the name and quantity of the portion of the pharmaceutical medication and the location for the portion of the pharmaceutical medication in the inventory; and (d) storing the portion of the pharmaceutical medication at the location in the inventory.

Further aspects of the present disclosure relate to methods for dispensing one or more pharmaceutical medications. In some embodiments, the methods comprise (a) receiving an order for one or more pharmaceutical medications, wherein the order indicates a name and a quantity for each of the one or more pharmaceutical medications; (b) at an electronic device with a processor and memory, inputting data related to at least a portion of the one or more pharmaceutical medications, wherein the data identify the name and quantity for the portion of the one or more pharmaceutical medications; (c) receiving a location identifier for at least a portion of the one or more pharmaceutical medications from the electronic device, wherein the location identifier identifies a location of the portion of the one or more pharmaceutical medications in an inventory, and wherein the ordering of pharmaceutical medications in the inventory is not alphabetical by pharmaceutical medication name; (d) retrieving at least a portion of the one or more pharmaceutical medications from the inventory based on the location identifier(s); and (e) dispensing at least a portion of the one or more pharmaceutical medications retrieved in (d) based on the received order.

Yet further aspects of the present disclosure relate to methods for dispositioning one or more pharmaceutical medications. In some embodiments, the methods comprise (a) at an electronic device with a processor and memory, inputting data identifying one or more of: an expiration date for one or more pharmaceutical medications and a name for one or more pharmaceutical medications (including, e.g., a medication identification code such as NDC); (b) receiving from the electronic device a location identifier for at least a portion of the one or more pharmaceutical medications, wherein the location identifier identifies a location of at least a portion of the one or more pharmaceutical medications in an inventory, and wherein the ordering of pharmaceutical medications in the inventory is not alphabetical by pharmaceutical medication name; (c) retrieving at least a portion of the one or more pharmaceutical medications from the inventory based on the location identifier; and (d) dispositioning at least a portion of the one or more pharmaceutical medications retrieved in (c).

In some embodiments of any of the above methods, the ordering of pharmaceutical medications in the inventory is not alphabetical by pharmaceutical medication name (e.g., brand, trade, and/or generic name). In some embodiments, the ordering of pharmaceutical medications in the inventory is not alphabetical, but is based on one or more of: a previous location in the inventory, a proximal location in the inventory, an available location in the inventory, an expiration date of the pharmaceutical medication, lot or batch number of the pharmaceutical medication, quantity of the pharmaceutical medication, packaging type of the pharmaceutical medication, the inventory turnover of the pharmaceutical medication, one or more storage requirements (e.g., temperature and/or humidity) of the pharmaceutical medication, and a condition of the pharmaceutical medication (e.g., whether the pharmaceutical medication is recalled, expired, suspect, illegitimate, or otherwise potentially unsafe). In some embodiments, an inventory is grouped by one or more the above, then ordered within each group non-alphabetically. In some embodiments, the ordering of medications in an individual aisle, area, shelving unit, shelf, inventory container or section may have some alphabetical ordering, but the overall ordering of pharmaceutical medications in the inventory is not alphabetical. In some embodiments, an inventory is grouped by medication source, expiration date, condition of the medication, and/or storage requirements for the medication, then optionally ordered within each group non-alphabetically.

Advantageously, these methods do not rely upon alphabetical sorting of drugs, but rather a “chaotic” storage system that uses a live inventory. These methods reduce the potential for errors in dispensing, since drugs with similar names are not stored together, and in storing, since drugs cannot be stored in inventory without creating or updating a record in live inventory and assigning a new location. These methods also allow for “pull-based” rather than “push-based” inventories, which reduce time burdens. Under existing methods, all incoming drugs must be sorted alphabetically for storage, even though many of them may not end up being dispensed and will expire on the shelf. Under the “pull-based” methods described herein, there is no time or labor cost spent organizing or sorting alphabetically when a shipment is received, thereby eliminating time wasted on sorting drugs that would ultimately expire. The methods described herein also allow for more efficient usage of inventory space, since incoming drugs can be added to any location in inventory, thus eliminating the need to reserve a dedicated shelf spot for a particular medication. These methods are particularly useful for inventories with donated or returned medications, since a pharmacy that receives donated or returned medications would have little control over the makeup or ordering of incoming medication shipments, exacerbating the problems of non-live inventory (based on traditional pharmacy inventory methods) due to a more unpredictable inventory. In some embodiments, this live inventory can help determine when it is appropriate to purchase or reorder medications, such as medications that are low or out-of-stock. In some embodiments, this live inventory can help determine when it is appropriate to delay the purchase or reorder of medications, such as medication that currently are expensive but may experience a price decline. In some embodiments, this live inventory can help determine when it is appropriate to transfer medications to or from another location, such as among stores or warehouses within a chain. In some embodiments, this live inventory can help determine when it is appropriate to transfer an order to or from another location, such as transferring a prescription from a first pharmacy to a second pharmacy because the first pharmacy was out-of-stock of an ordered medication.

In some embodiments, alphabetical ordering refers to an inventory that is grouped by storage requirement, expiration date, source, condition, packaging type, dosage form, route of administration, and/or another characteristic, then ordered within each group alphabetically. In some embodiments, alphabetical ordering refers to an inventory that is ordered alphabetically without first grouping medications, e.g., by dosage form, route of administration, and/or another characteristic.

As used herein, the terms “pharmaceutical medication” and “drug” can be used interchangeably and may refer to any therapeutic pharmaceutical substance or pharmaceutical medications (e.g., including prescription and over-the-counter or non-prescription medications), as well as medical supplies, pharmaceutical ingredients, and pharmaceutical precursors.

As used herein, unless clearly indicated otherwise by context, a medication or drug “name” can refer to a brand name, generic or non-proprietary name, or a trade name. In addition to the brand name, generic or non-proprietary name, or trade name, the name can further comprise a strength, dosage, and/or dosage form (e.g., esomeprazole 20 mg vs. 40 mg, or esomeprazole delayed-release capsules vs. esomeprazole sodium for injection/infusion). “Name” also includes any data from which the “name” could be looked up, derived, or inferred, regardless of whether such action is taken, including without limitation a Prescription Number, NDC code, or Product Identifier.

As used herein, unless clearly indicated otherwise by context, a medication or drug “quantity” can refer to the number of pills (e.g., tablets, capsules, caplets, half-tablets), units (e.g., syringes, patches, inhalers), volume (e.g., milliliters), strength (e.g., milligrams), uses (e.g., injections, length of time, inhalations). “Quantity” also includes any data from which the “quantity” could be looked up, derived, or inferred, regardless of whether such action is taken, including without limitation a Prescription Number, Package Size (e.g., NDC Packaging Code), Container Size, Product Identifier.

As used herein, a “medication identification code” can refer to a code according to any coding system used as a shorthand for one or more pieces of information about a pharmaceutical medication. For example, a medication identification code can be any code (e.g., alphanumeric code) used to label a drug in the inventory. A medication identification code can comprise any format, e.g., one or both of a human readable or machine readable format (including but not limited to an optical barcode or RFID). A medication identification code can include, identify, or indicate one or more of: Prescription Number (Rx), serial number, lot or batch number, National Drug Code (NDC), Universal Product Code (UPC), International Article Number (EAN), Generic Product Identifier (GPI), Generic Code Number (GCN), Generic Sequence Number (GSN), Product Identifier, or a customized code.

As used herein, “inputted data” can comprise any inputted information associated with at least a portion of at least one pharmaceutical medication. For example inputted data may include one or more of: an expiration date of the pharmaceutical medication, source (e.g. wholesaler, repackager, health facility, pharmacy, third party logistics provider), medication identification code such as an NDC, strength, dosage, dosage form, manufacturer, labeler, lot or batch number of the pharmaceutical medication, quantity of the pharmaceutical medication, packaging size or type of the pharmaceutical medication, storage requirement of the pharmaceutical medication (e.g., temperature and/or humidity), pharmaceutical medication source, tracking information, shipment method, courier, repacked by user, reconciled by user, created by user, inputted by user, updated by user, shipped by user, received by user, stored by user, pended by user, retrieved by user, dispensed by user, dispositioned by user, date repacked, date reconciled, date created, date inputted, date updated, date shipped, date received, date stored, date pended, date retrieved, date dispensed, date dispositioned, transactional information or history, and condition of the pharmaceutical medication (e.g., whether the pharmaceutical medication is recalled, expired, suspect, illegitimate, or otherwise potentially unsafe). In some embodiments, only a portion or segment of an medication identification code is inputted (e.g., the labeler and product code from the first two segments of an NDC). In some embodiments, the NDC segment(s) may be padded with the digit 0 and/or may or may not use a delimiter such as a dash (“-”) to differentiate between segments. In some embodiments, the inputted data can be calculated or derived automatically. For example, the electronic device may provide the current date, an identifier of the user who is currently logged in, or look up input data such as the cost, price, tracking, the inventory turnover, or transaction information from a database, documentation, or Application Programming Interface (API). In another example, the electronic device could calculate a previous location in the inventory, a proximal location in the inventory, an available location in the inventory.

The methods of the present disclosure can be applied to donated medication(s), purchased medication(s), and/or returned medication(s), and combinations thereof. For further description of donated medications and systems and methods related thereto, see U.S. Pat. No. 9,892,433. A medication donation may refer to the transfer of a medication from a donor to a recipient. In some implementations, the terms donation/donate may refer to giving a medication to a recipient in-kind without fee. In some implementations, the terms donation/donate may include providing a medication to a recipient in exchange for a nominal payment, such as a payment at less than market cost. In some implementations, the terms donation/donate may refer to receiving a medication in exchange for a fixed “membership” fee paid by the donor or recipient, or a fee based on usage rather than discrete donations and/or medications (e.g., charging a fee based on the number of donors in the system, number of donations made, etc.).

### I. Methods for Storing a Pharmaceutical Medication

Certain aspects of the present disclosure relate to methods for storing a pharmaceutical medication in an inventory.

In some embodiments, the methods comprise (a) receiving a pharmaceutical medication; (b) inputting (e.g., at an electronic device of the present disclosure with a processor and memory) data related to the pharmaceutical medication, wherein the data identify: (1) name and quantity of at least a portion of the pharmaceutical medication, and (2) a location for at least a portion of the pharmaceutical medication in an inventory, wherein the ordering of pharmaceutical medications in the inventory is not alphabetical by pharmaceutical medication name; (c) creating a record associated with at least a portion of the pharmaceutical medication in the memory of the electronic device, wherein the record identifies the name and quantity of the portion of the pharmaceutical medication and the location for the portion of the pharmaceutical medication in the inventory; and (d) storing the portion of the pharmaceutical medication at the location in the inventory.

In some embodiments, the data (e.g., inputted data) comprise name and/or quantity of the pharmaceutical medication. In some embodiments, the data identify name and/or quantity of the pharmaceutical medication, e.g., by comprising information including but not limited to a medication identification code such as a National Drug Code (NDC), which could then be used to derive the name and/or quantity. As such, the inputted data can identify the name and quantity, or could be used to identify the name and quantity (whether or not the name and quantity is directly inputted or derived).

In some embodiments, inputted data related to a received pharmaceutical medication can be derived, at least in part, from documentation associated with the received pharmaceutical medication. For example, the documentation may be physical and/or electronic. In some embodiments, the documentation comprises one or more of medication packaging, order invoice(s), order receipt(s), shipping manifest(s), packing slip(s), transaction statement(s), transaction information, and transaction history. In some embodiments (e.g., as with electronic documentation), inputted data may be looked up, derived, or processed, at least in part, using one or more centralized or distributed databases run by one or more organizations or regulatory agencies, Neural Networks, Deep Learning, and/or Artificial Intelligence (AI), e.g., using an electronic device of the present disclosure.

In some embodiments, the data related to the pharmaceutical medication are inputted into the electronic device manually, e.g., by typing or other human-performed data entry. In some embodiments, the data related to the pharmaceutical medication are inputted into the electronic device via an optical barcode scanner, RFID reader, Optical Character Recognition (OCR), Natural Language Processing (NLP), or other automated or semi-automated data entry system. For example, some or all of the data (e.g., name, quantity, and/or location in the inventory) can be inputted into the electronic device via an optical barcode scanner or other automated data entry system.

In some embodiments, the methods further comprise (e.g., after storing the portion of the first pharmaceutical medication in the inventory): receiving a second shipment comprising the pharmaceutical medication; inputting (e.g., at the electronic device) second data related to the pharmaceutical medication, wherein the second data identify: name of the pharmaceutical medication and quantity of at least a portion of the pharmaceutical medication received in the second shipment, and the location for at least a portion of the pharmaceutical medication in the inventory; updating the record associated with the pharmaceutical medication in the memory of the electronic device; and storing the portion of pharmaceutical medication received in the second shipment at the location in the inventory. In some embodiments, the methods further comprise (e.g., after storing the portion of the pharmaceutical medication in the inventory): receiving a second shipment comprising the pharmaceutical medication; inputting (e.g., at the electronic device) second data related to the pharmaceutical medication, wherein the second data identify: name of the pharmaceutical medication and quantity of at least a portion of the pharmaceutical medication received in the second shipment, and the location for at least a portion of the pharmaceutical medication in the inventory; creating a record associated with the pharmaceutical medication received in the second shipment in the memory of the electronic device; and storing the portion of pharmaceutical medication received in the second shipment at the location in the inventory. As will be understood by one of skill in the art, references herein to updating or creating a new record in memory of an electronic device can be interchangeable, depending on specific database design. In some embodiments, the updated or created record further identifies a quantity of the pharmaceutical medication based at least in part on the quantity of at least a portion of the pharmaceutical medication received in the second shipment. For example, the updated or created record may identify a combined quantity of the pharmaceutical medication (e.g., at the location in the inventory) based on two or more shipments (if some or all of the medication(s) in the first shipment remain in the inventory). In some embodiments, e.g., if the medication(s) from the first shipment has already been dispensed or disposed, the updated or created record identifies a quantity of the pharmaceutical medication based on the quantity of the pharmaceutical medication received in the second shipment. Advantageously, these processes allow for a live inventory of the pharmaceutical medication in the inventory.

In some embodiments, the methods further comprise (e.g., after storing the portion of the pharmaceutical medication in the inventory): receiving a second pharmaceutical medication; inputting (e.g., at the electronic device) second data related to the second pharmaceutical medication, wherein the second data identify: name of the second pharmaceutical medication and quantity of at least a portion of the second pharmaceutical medication received, and the location for at least a portion of the second pharmaceutical medication in the inventory; creating a record associated with the second pharmaceutical medication received in the memory of the electronic device; and storing the portion of the second pharmaceutical medication received at the location in the inventory.

In some embodiments, the updated or created record further identifies a quantity of the pharmaceutical medication based at least in part on the quantity of at least a portion of the second pharmaceutical medication received. For example, the updated or created record may identify a combined quantity of two or more pharmaceutical medications (e.g., at the location in the inventory) received within the same shipment. For example, the updated or created record may identify a combined quantity of two or more pharmaceutical medications (e.g., at the location in the inventory) received within different shipments. In some embodiments, e.g., if the first medication(s) have already been dispensed or dispositioned, the updated or created record identifies a quantity of the pharmaceutical medication based on the quantity of the second pharmaceutical medication received

In some embodiments, the methods further comprise (e.g., after storing the portion of the pharmaceutical medication in the inventory): receiving a second shipment comprising the pharmaceutical medication; inputting (e.g., at the electronic device) second data related to the pharmaceutical medication, wherein the second data identify: name of the pharmaceutical medication and quantity of at least a portion of the pharmaceutical medication received in the second shipment, and a second location for at least a portion of the pharmaceutical medication in the inventory; updating the record associated with the pharmaceutical medication in the memory of the electronic device, wherein the updated record further identifies the second location for the pharmaceutical medication in the inventory and a quantity of the pharmaceutical medication based at least in part on the quantity of the portion of the pharmaceutical medication received in the second shipment; and storing the portion of the pharmaceutical medication received in the second shipment at the second location in the inventory. In some embodiments, the methods further comprise (e.g., after storing the portion of the pharmaceutical medication in the inventory): receiving a second shipment comprising the pharmaceutical medication; inputting (e.g., at the electronic device) second data related to the pharmaceutical medication, wherein the second data identify: name of the pharmaceutical medication and quantity of at least a portion of the pharmaceutical medication received in the second shipment, and a second location for at least a portion of the pharmaceutical medication in the inventory; creating a second record associated with the pharmaceutical medication received in the second shipment in the memory of the electronic device, wherein the created record identifies the second location for the pharmaceutical medication in the inventory and a quantity of the pharmaceutical medication based at least in part on the quantity of the portion of the pharmaceutical medication received in the second shipment; and storing the portion of the pharmaceutical medication received in the second shipment at the second location in the inventory. In some embodiments, the second location of the pharmaceutical medication in the inventory is different from the first location of the pharmaceutical medication in the inventory (e.g., a different physical location, or a different location relative to one or more other medications in the inventory).

As such, the methods of the present disclosure allow for storing the same type of medication(s) received in the same or different shipments (e.g., at different times or part of different physical shipments) at different inventory locations. This differs from an alphabetical ordering of the inventory, in which the same drug is always stored at the same location (e.g., on a shelf, inventory container, etc.) in the inventory upon receipt. In some embodiments, quantities of the same, similar, or different pharmaceutical medications may be stored at the same location in some instances but at a different location in other instances. For example, completely different medications could be stored in the same location simply because that is the most convenient location for the person storing the medications. For example, quantities of the pharmaceutical medication with the same or similar expiration date, inventory turnover (e.g., fast-movers), serial number, or lot or batch number can be stored at the same location, but another quantity with a different expiration date, serial number, or lot or batch number can be stored at a different location. This may be advantageous, e.g., in prioritizing the use of earlier expiring medications first, dispensing fast moving medication quickly, or in segregating similar lots of a medication in case of recall or discontinuation.

In some embodiments, the inputted location for the pharmaceutical medication in the inventory is different from a previous location for a pharmaceutical medication having the same name, medication identification code such as an NDC, or medication identification code as the received pharmaceutical medication. In some embodiments, the location is a precise physical location. In some embodiments, the location is a location relative to one or more drugs (for example, under alphabetical ordering, the precise physical location of a drug on the shelf may move if a drug with an earlier alphabetical name is added to the inventory, but the relative location of the drug is the same, since the drugs before and after it in the inventory remain constant). This differs from alphabetical ordering, in which the precise physical or relative location of a medication is always the same. As such, these methods allow the same medication to be stored in different locations in the inventory.

In some embodiments, the methods further comprise (e.g., after or concurrent with creating a record) updating the record (e.g., in the memory of the electronic device) to identify a received portion of a pharmaceutical medication as stored. In some embodiments, a more specific marking may indicate a stored status. In some embodiments, these more specific markings may indicate the status as, e.g., available, received, shipped, verified, accepted, logged, entered, on-hand, in-stock, low-stock, high-stock, and the like. This informs the user as to the current status of the medication in a live inventory.

In some embodiments, the methods further comprise associating a code with the pharmaceutical medication (e.g., such that the code is associated with the medication in the inventory). For example, a code can be affixed to the packaging of the medication, or placed at a location proximal to the medication in the inventory. In some embodiments, the code comprises an optical barcode or radio frequency identification (RFID) code. In some embodiments, the code is associated after receiving the medication, inputting data, and/or creating a record. In some embodiments, the code is associated after or concurrently with storing the medication in the inventory. In some embodiments, the code is associated prior to or concurrently with storing the medication in the inventory. In some embodiments, the code is derived at least in part from inputted data. In some embodiments, the code is a randomly generated and/or unique code which is associated with the inputted data. For example, upon receipt, a pharmaceutical medication could be associated with a pre-printed code by entering the pre-printed code as part of the inputted data thereby linking the code with the medication. Alternatively, after inputting data and/or creating a record, an electronic device of the present disclosure can generate a new code associated with the medication based on the inputted data.

In some embodiments, data related to the pharmaceutical medication or location are inputted (e.g., at the electronic device) through an optical barcode scanner, RFID reader, or OCR device.

In some embodiments, the location for the pharmaceutical medication in the inventory is automatically inputted by the electronic device, e.g., based on one or more criteria. In some embodiments, the criteria include, without limitation, and in any combination or weighting schema, a previous location in the inventory, a proximal location in the inventory, an available location in the inventory, and inputted data of the present disclosure.

Advantageously, this allows the device to determine an optimized location for a pharmaceutical medication in the inventory based on predetermined criteria of interest. For example, medications that are often prescribed together can be located near one another in the inventory, reducing the amount of time needed to fill a common order. Medications can be grouped by expiration date or serial or lot or batch number which could aid in dispositioning expired and recalled medications. Medications with different names but similar storage requirements can be stored under the same conditions. Medications can be stored in different ways in the inventory based upon size and/or type of packaging. These features allow for an inventory with higher storage density, improved accuracy (e.g., by putting dissimilar medications together), quicker fill times (e.g., by optimizing fulfillment route), and no requirement for memorization of which drugs go where (which reduces the potential for error). In addition, these features allow for easier reorganization of the inventory, as simply changing the criteria can allow the device to direct the user to store medications at a different location (which is less confusing than always storing the medications in one location, memorizing it, then having to change the location and remember that the location has changed). In certain embodiments, the criteria include a previous location in the inventory, a proximal location in the inventory, and/or an available location in the inventory.

In some embodiments, the inventory can include one or more locations for quarantined medication(s), or medication(s) otherwise marked for disposition (e.g., based on condition, expiration date, etc.). In some embodiments, an empty or placeholder location identifiers can be used to designate a mandatory, preferred or suggested location in the inventory for the medication(s) to be stored, e.g., an empty location may be designated for quarantining medication(s) that are ready for disposition. In some embodiments, a location identifier four characters long could be for large locations divided into sections (ideal for large or abnormally shaped items such a bubble packed medications). In some embodiments, a location identifier three characters long could be for small locations that are not divided into sections (ideal for small or uniformly shaped items such as repacked medications). In some embodiments, a location identifier starting with an ‘X’ could mean pending medications which are ready to be retrieved, repacked, or dispensed. In some embodiments, a location identifier containing ‘UNIT’ might be for unit-of-use medications such as inhalers, patches and/or injections. In some embodiments, a location identifier could contain one or more characters that represent which shelf in the shelving unit that location, location identifier could contain one or more characters that represent which location within the shelf of that location. For example the location identifier “RB123” could mean the 3^{rd} section within an inventory container that is the 2^{nd} inventory container located on the 1^{st} shelf of Shelving Unit B in the ‘R’efrigerated Area.

In some embodiments, the methods further comprise associating an expiry indicator with the pharmaceutical medication (e.g., such that the expiry indicator is associated with the medication in the inventory). For example, an expiry indicator can be affixed to the packaging of the medication, or placed at a location proximal to the medication in the inventory. In some embodiments, the code is associated after receiving the medication. In some embodiments, the expiry indicator is associated after or concurrently with storing the medication in the inventory. In some embodiments, the expiry indicator is associated prior to storing the medication in the inventory. In some embodiments, the expiry indicator indicates a date, month, or year of expiry (e.g., based on the medication expiration date), such as by color-coding, thus making it easy to identify medication(s) having a similar expiration date in the inventory (e.g., by looking for a purple sticker that says “August” or “2018”). This provides a more visually recognizable way to identify an expiration date for a medication in the inventory without requiring a user to read the actual expiration date on the packaging, which can be more difficult to see and/or immediately recognize.

In some embodiments, a pharmaceutical medication (or portion thereof) is stored in an inventory container. Any container suitable for storing a pharmaceutical medication can be used. In some embodiments, the inventory container is portable and can be taken to the data entry workstation, which can reduce or eliminate the need of the person receiving and/or storing medications to move around while restocking inventory. In some embodiments, the inventory container comprises a plurality of sections, e.g., in which one or more section(s) of the plurality are labeled with a different location in the inventory. In some embodiments, the sections of the plurality are divided by physical divider(s), e.g., within the inventory container. In some embodiments, the physical divider(s) are movable, e.g., within the inventory container. Advantageously, this allows flexibility in the types, packaging, dosage forms, and/or quantities of pharmaceutical medications that can be stored in the sections and containers.

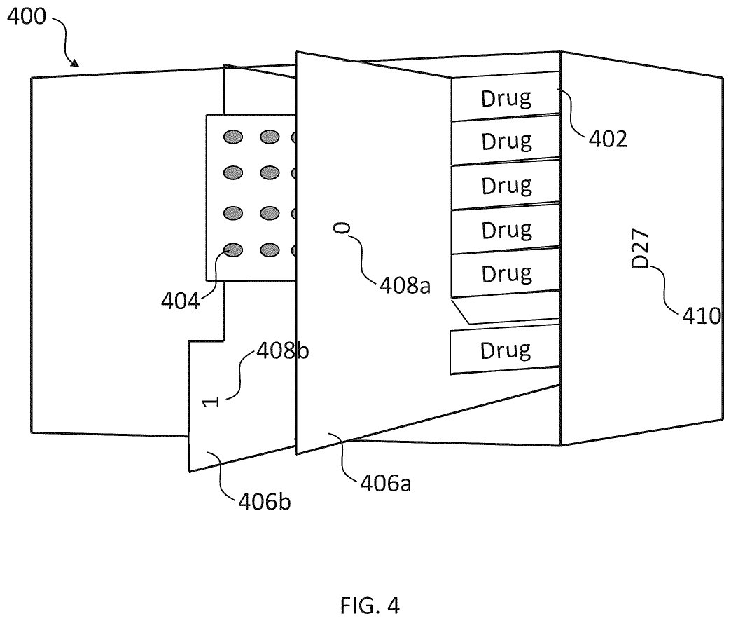

FIG. 4 illustrates exemplary inventory container 400. In this example, container 400 is labeled with indication of an inventory container 410 (labeling the inventory container as “D27”). Indication 410 can thus indicate container 400's location in the inventory, in this example Shelving Unit D, Shelf 2, Row 7. Stored in container 400 are pharmaceutical medications 402 (in pharmaceutical packages) and 404 (in a blister pack). As shown, medications 402 and 404 are stored in different sections of container 400. These sections are divided by divider 406a, just as one or more additional sections are divided from the section of drug 404 by divider 406b. Dividers 406a and 406b are further labeled with section indications 408a and 408b, respectively. Advantageously, dividers 406a and 406b are movable within container 400, allowing the sizes of the sections holding medications 402 and 404 to be flexibly adjusted, e.g., depending upon the size/quantity of the medications and their associated packaging. In this example, the location of medication 402 can be indicated as “D27-0” or simply “D270” and the location of medication 404 as “D27-1” or simply “D271”.

In some embodiments, at least a portion of the inventory is at room temperature. In some embodiments, at least a portion of the inventory is cooler than room temperature, e.g., refrigerated.

In some embodiments, a location in the inventory at which a medication is stored identifies one or more of: an aisle or area, a shelving unit, an inventory container, and a section within an aisle, area, shelving unit, or inventory container. In some embodiments, the inventory comprises a plurality of sections, and each section of the plurality is labeled with a different location in the inventory. In some embodiments, a sub-location within the inventory (e.g., an aisle or area, a shelving unit, or inventory container) comprises a plurality of sections, and each section of the plurality is labeled with a different location in the inventory.

In some embodiments, sections in the inventory are divided by physical dividers. In some embodiments, one or more of the physical dividers is movable within the inventory. For example, an aisle or area, a shelving unit, or inventory container can contain one or more movable, physical dividers, thereby allowing flexibility for storage by enabling sections of adjustable size/dimensions. In some embodiments, one or more of the physical dividers are movable directly by hand. In some embodiments, one or more of the physical dividers are movable via notches, spring, screw, motor, gravity, or another mechanically based mechanism.

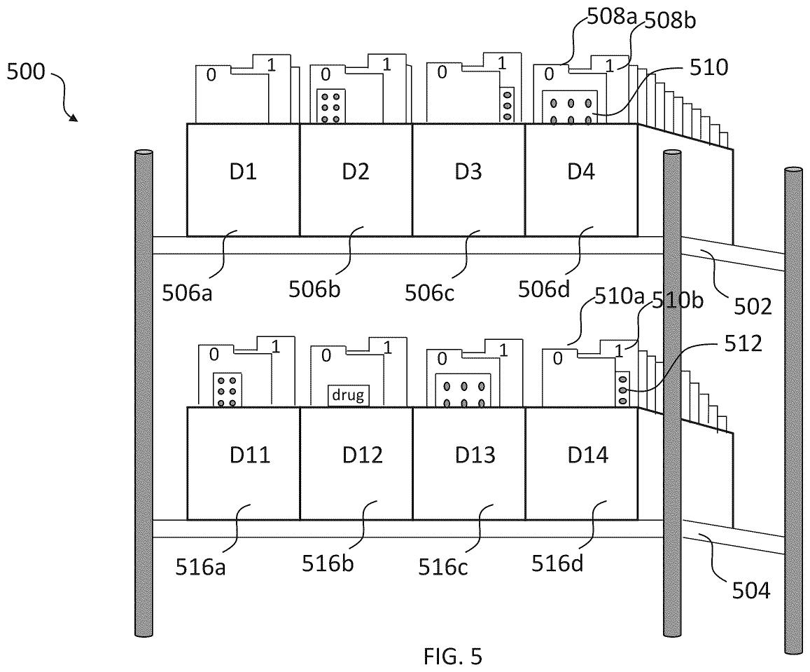

FIG. 5 illustrates exemplary inventory shelving unit 500. As shown, shelving unit 500 includes shelves 502 and 504, each holding a row of inventory containers. Inventory containers 506a-d are placed on shelf 502, and containers 516a-d on shelf 504. Each inventory container is labeled with an indicator (e.g., “D1” on 506a). These indicators provide an indication of a shelving unit (“D” for unit 500), an indication of a shelf (“1-4” for shelf 502 and “11-14” for shelf 504). Inventory containers 506a-d and 516a-d also comprise a plurality of dividers, such as dividers 508a and 508b in container 506d, and dividers 510a and 510b in container 516d. As discussed above in reference to FIG. 4, these dividers can demarcate sections in each container. The dividers are also labeled with indications of a section (e.g., “0” on 508a, indicating that drug 510 is in section “0” of inventory container “D4” on shelving unit 500). The containers further contain medications stored in the inventory, such as medications 510 and 512.

In some embodiments, the methods further comprise (e.g., after creating a record) storing in the record an indication of an expiration date and/or lot or batch number of the pharmaceutical medication. For example, the expiration date and/or lot or batch number can be entered with, looked up, derived, or indirectly inferred (e.g., by the device or by a user) from, the inputted data. For example, the inputted data can further indicate a medication identification code such as a Product Identifier of the pharmaceutical medication, and the expiration date, lot or batch number, and/or quantity can be looked up or derived from the Product Identifier.

In some embodiments, the inputted data further identifies one or more of: pharmaceutical medication source, tracking information, transactional information, and transactional history. For example, the tracking/transactional information/history can be based on shipping information related to the shipment in which the medication was received. In some embodiments, scanning the tracking code, could retrieve the necessary documentation associated with all the medications received in the shipment. In some embodiments, this documentation could then be automatically entered into the electronic device prior to or concurrently with creating or updating one or more records associated with the medications in the shipment.

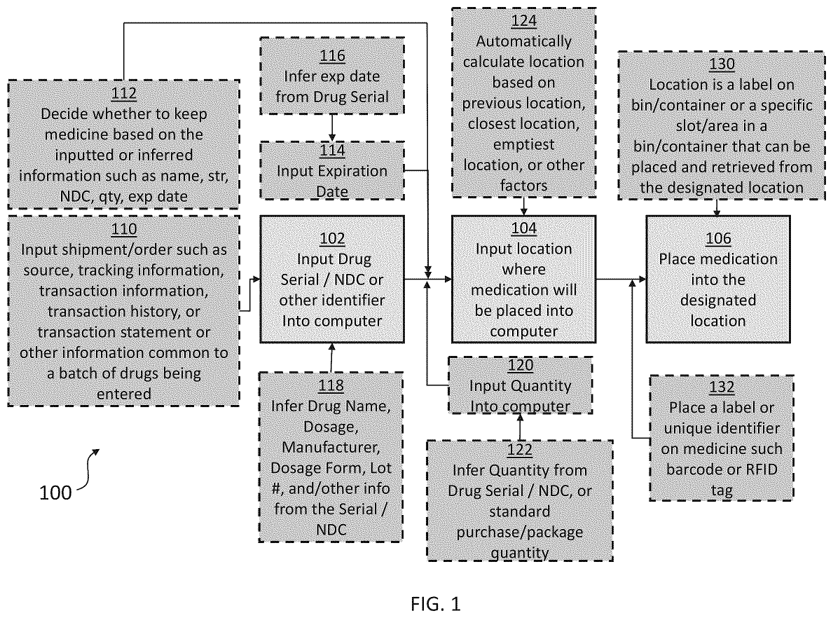

FIG. 1 provides a flowchart for exemplary process 100 that may be used in the methods for storing a pharmaceutical medication in an inventory of the present disclosure.

At block 102, data identifying the name, medication identification code such as an NDC of a pharmaceutical medication are inputted at a computer. Optionally, at block 110, data related to a shipment or order are inputted, e.g., identifying the source, tracking information, transaction information, transaction history, transaction statement, or other information common to a batch or shipment of drugs being entered. Optionally, at block 112, a user decides whether to keep a medication, e.g., based on inputted data and/or information inferred therefrom (such as name, medication identification code, quantity, expiration date, etc.). Optionally, at block 114, expiration date for one or more medication(s) is inputted. Alternatively, at block 116, an expiration date can be inferred, e.g., from a serial number of the medication, by the user or the device. Optionally, at block 118, information such as name, dosage, manufacturer, labeler, dosage form, lot or batch number, and the like can be inferred, e.g., from a medication identification code such as an NDC, by the user or the device. Optionally, at block 120, quantity is inputted. Alternatively, at block 122, quantity can be inferred (e.g., from a medication identification code, standard or previous order, or package size/quantity) by the user or the device.

At block 104, data identifying a location for at least a portion of the pharmaceutical medication are inputted at a computer. Data inputted at blocks 102 and 104 can be used to create a store a record identifying the name, quantity, and location of the medication. Optionally, at block 124, the location is calculated or suggested based on, e.g., previous location, proximal location, emptiest location, and so forth. Optionally, at block 130, the location is a labeled bin or container, or a specific section in a bin or container. A bin or container can be moved within, placed in, and/or retrieved from the inventory.

At block 106, at least a portion of the medication is stored in the location. Optionally, at block 132, a label or unique identifier (e.g., a barcode or RFID tag) is associated with the medication.

In some embodiments, one or more of the steps or processes in the methods for storing a pharmaceutical medication in an inventory of the present disclosure may be combined, in any number, order, or combination, with one or more of the steps or processes in the methods for dispensing and/or dispositioning a pharmaceutical medication of the present disclosure. For example, in some embodiments, the methods of the present disclosure comprise (a) receiving a pharmaceutical medication; (b) at an electronic device with a processor and memory, inputting data related to the pharmaceutical medication, wherein the data identify: (1) name and quantity of at least a portion of the pharmaceutical medication, and (2) a location for at least a portion of the pharmaceutical medication in an inventory; (c) creating a record associated with at least a portion of the pharmaceutical medication in the memory of the electronic device, wherein the record indicates the name and quantity of the portion of the pharmaceutical medication and the location for the portion of the pharmaceutical medication in the inventory; (d) storing the portion of the pharmaceutical medication at the location in the inventory; (e) receiving an order for at least a portion of at least one of the pharmaceutical medications received in (a), wherein the order identifies a name and a quantity for the portion of at least one of the pharmaceutical medications; (f) at the electronic device, inputting data related to at least a portion of at least one of the pharmaceutical medications, wherein the data identify the name and quantity for the portion of at least one of the pharmaceutical medications; (g) receiving a location identifier for at least a portion of at least one of the pharmaceutical medications from the electronic device, wherein the location identifier identifies the location of the portion of at least one of pharmaceutical medications in the inventory; (h) retrieving at least a portion of at least one of the pharmaceutical medications from the inventory based on the location identifier(s); and (i) dispensing at least a portion of at least one of the pharmaceutical medications retrieved in (h) based on the received order.

In some embodiments, the methods of the present disclosure comprise (a) receiving a pharmaceutical medication; (b) at an electronic device with a processor and memory, inputting data related to the pharmaceutical medication, wherein the data identify: (1) name and quantity of at least a portion of the pharmaceutical medication, and (2) a location for at least a portion of the pharmaceutical medication in an inventory; (c) creating a record associated with at least a portion of the pharmaceutical medication in the memory of the electronic device, wherein the record indicates the name and quantity of the portion of the pharmaceutical medication and the location for the portion of the pharmaceutical medication in the inventory; (d) storing the portion of the pharmaceutical medication at the location in the inventory; (e) at an electronic device with a processor and memory, inputting data identifying one or more of: an expiration date for one or more pharmaceutical medications, a medication identification code such as an NDC for one or more pharmaceutical medications, and a name for one or more pharmaceutical medications; (f) receiving from the electronic device a location identifier for at least a portion of at least one of the pharmaceutical medications, wherein the location identifier identifies a location of at least a portion of at least one of the pharmaceutical medications in an inventory, and wherein the ordering of pharmaceutical medications in the inventory is not alphabetical by pharmaceutical medication name; (g) retrieving at least a portion of at least one of the pharmaceutical medications from the inventory based on the location identifier; and (h) dispositioning at least a portion of at least one of the pharmaceutical medications retrieved in (g).

In some embodiments of any of the above methods, the ordering of pharmaceutical medications in the inventory is not alphabetical by pharmaceutical medication name (e.g., brand, trade, generic name, and/or dosage).

### II. Methods for Dispensing a Pharmaceutical Medication

Certain aspects of the present disclosure relate to methods for dispensing a pharmaceutical medication.

In some embodiments, the methods comprise (a) receiving an order for one or more pharmaceutical medications, wherein the order indicates a name and a quantity for each of the one or more pharmaceutical medications; (b) at an electronic device with a processor and memory, inputting data related to at least a portion of the one or more pharmaceutical medications, wherein the data identify the name and quantity for the portion of the one or more pharmaceutical medications; (c) receiving a location identifier for at least a portion of the one or more pharmaceutical medications from the electronic device, wherein the location identifier identifies a location of the portion of the one or more pharmaceutical medications in an inventory, and wherein the ordering of pharmaceutical medications in the inventory is not alphabetical by pharmaceutical medication name; (d) retrieving at least a portion of the one or more pharmaceutical medications from the inventory based on the location identifier(s); and (e) dispensing at least a portion of the one or more pharmaceutical medications retrieved in (d) based on the received order. In some embodiments, e.g., if the order is for more than one pharmaceutical medication, a portion (or all) of each of the medications in the order is dispensed, or a portion (or all) of only one of the medications in the order is dispensed.

As used herein, dispensing a medication can include one or both of providing at least a portion of the pharmaceutical medication directly to a patient or distributing the pharmaceutical medication (or at least a portion thereof) through one or more intermediaries and/or third parties, which then provide at least a portion of the pharmaceutical medication to a patient.

In some embodiments, the one or more pharmaceutical medications are non-prescription or over-the-counter medications. In some embodiments, the order identifies the name(s), strength(s), dosage form(s), and/or quantity of the medications.

In some embodiments, the one or more pharmaceutical medications are prescription medications. For example, the order can comprise a prescription or refill for the medications, e.g., identifying the name(s), strength(s), dosage forms(s), and/or quantity of the medications. In some embodiments, the refill may be requested by the patient. In some embodiments, the refill may be provided by the prescriber. In some embodiments, the refill may be scheduled, for example, as part of a pharmacy autofill program that automatically sends refills at predetermined intervals.

In some embodiments, the order can also include receiving information about the patient, such as a physical or electronic registration form and/or a patient profile from a pharmacy benefit manager. In some embodiments, this patient information can be received before or after the order. In some embodiments, this information is stored as a record on an electronic device. In some embodiments, the registration form may include one or more of patient name, gender, date of birth, contact information—e.g., phone number(s), email address(es), shipping address(es)—username, password, billing address(es), insurance information, medication allergies, medical history, medical conditions, a list of other drugs being taken, payment information, and/or HIPAA consent. In some embodiments, the patient and/or their insurance is billed for the order based on the insurance and/or payment information provided in the order.

In some embodiments, an order is received at an electronic device of the present disclosure automatically. In some embodiments, the data related to the one or more pharmaceutical medications are inputted automatically at the electronic device.

In some embodiments, a generic or therapeutic equivalent of the medication(s) is dispensed. For example, a location identifier received from an electronic device of the present disclosure can identify the location of a generic or therapeutic equivalent of the one or more pharmaceutical medications from a received order, and the equivalent (or a portion thereof) can be retrieved from the inventory and dispensed.

In some embodiments, the location for the pharmaceutical medication in the inventory received from an electronic device of the present disclosure is based on one or more criteria. In some embodiments, the criteria include, in any combination or weighting schema, inputted data of the present disclosure. For example, the device can provide a location for the medication based on proximity to another medication in the order, location based on a future (and not past) expiration date, location for capsules and not tablets. This provides a live inventory that can be utilized efficiently (e.g., by reducing medication waste due to expiry, or by reducing fulfillment time during retrieval and dispensing).

For example, the device can provide a location for the medication based on proximity to another medication in the order, location based on a future (and not past) expiration date, location for capsules and not tablets. This provides a live inventory that can be utilized efficiently (e.g., by reducing medication waste due to expiry, or by reducing fulfillment time during retrieval and dispensing). In certain embodiments, the criteria include a previous location in the inventory, a proximal location in the inventory, and/or an available location in the inventory.

In some embodiments, a received order is for two or more pharmaceutical medications in the inventory (e.g., two or more, three or more, four or more, five or more, six or more, seven or more, eight or more, nine or more, ten or more, and so forth). For example, the order (or a second received order) can include information related to a second pharmaceutical medication (e.g., identifying or indicating the second medication's name, quantity, expiration date, and/or medication identification code such as an NDC). In some embodiments, the received order is for one pharmaceutical medication, and fulfillment of the order requires retrieving the pharmaceutical medication from two or more locations in the inventory. For example, an order could be for a quantity of 100, so the user needs to access 4 separate locations, each containing a quantity of 25. In some embodiments, the methods further comprise (e.g., after inputting data related to the one or more pharmaceutical medications at the electronic device) receiving from the electronic device (1) two or more location identifiers of at least a portion of one or more pharmaceutical medications, wherein each of the two or more location identifiers identifies the location of at least a portion of one or more pharmaceutical medications in the inventory, and (2) a suggested path for retrieving at least a portion of the one or more pharmaceutical medications from the inventory (e.g., some or all of the medications in the order(s), including portions thereof) based on the two or more location identifiers; retrieving at least a portion of the one or more pharmaceutical medications from the inventory according to the suggested path; and dispensing or dispositioning the retrieved portion of the one or more pharmaceutical medications. In some embodiments, the suggested path provides a walking path through the inventory for retrieving the medications. In some embodiments, the suggested path provides a suggested order for retrieving the medications in the inventory (e.g., an ordered “pick list”). In some embodiments, the suggested path provides the shortest distance, or an optimized order of retrieval that allows for the shortest distance, to retrieve some or all of the medications from the inventory. In some embodiments, the suggested path identifies two or more locations for the same pharmaceutical medication. Advantageously, this allows the user to retrieve some or all of the medications in an order or orders using an optimized route in the inventory, thereby saving time and labor.

In some embodiments, retrieving the pharmaceutical medication comprises retrieving the whole shelving unit, shelf, inventory container, or section containing the pharmaceutical medication. In some embodiments, one or more pharmaceutical medications (or the shelving unit, shelf, inventory container, or section containing the medication) are retrieved from the inventory manually. For example, a pharmacist or technician can physically enter the inventory and retrieve pharmaceutical medication(s). In some embodiments, one or more pharmaceutical medications (or the shelving unit, shelf, inventory container, or section containing the medication) are retrieved from the inventory using an automated device. For example, the pharmaceutical medication(s) can be moved using a robot, conveyor, or other automated device.

In some embodiments, retrieving one or more pharmaceutical medications from the inventory comprises scanning a code associated with the pharmaceutical medication(s) and/or the location. For example, the code can comprise an optical barcode or RFID code.

In any of the methods described herein, and at any position or order in the processes of the methods described herein, one or more pharmaceutical medications can be repacked, e.g., one or more times. As used herein, repacking refers to modifying the packaging and/or labeling of one or more pharmaceutical medications, optionally modifying a quantity of the pharmaceutical medication(s) in a single package or container. In some embodiments, repacking comprises combining at least a portion of multiple packages of pharmaceutical medications into one package of pharmaceutical medication. In some embodiments, repacking comprises splitting at least a portion of one package of pharmaceutical medication into multiple packages of pharmaceutical medications. In some embodiments, repacking comprises combining or compounding multiple pharmaceutical ingredients or precursors into a pharmaceutical medication. For example, one or more pharmaceutical medications can be transferred from a blister pack or stock bottle to an amber vial (e.g., for dispensing to a patient) or shipping container (e.g., for dispensing to an intermediary or third party). In another example, upon receipt, pharmaceutical medication(s) can be repacked into a container for holding a different quantity (e.g., transferring from one or more blister packs into a vial or other container with a predetermined quantity, such as a count of 30 or other quantity of the medication that can be typically dispensed as part of a prescription). In some embodiments, one or more pharmaceutical medications are repacked after receipt, but before storing. In some embodiments, one or more pharmaceutical medications are repacked after retrieval, but before dispensing or dispositioning. In some embodiments, the repacking is done by a third party such as by a repackager or relabeler. In some embodiments, a second portion of one or more pharmaceutical medications are repacked upon or after a first portion is dispensed or dispositioned. In some embodiments, one or more pharmaceutical medications are repacked prior to receiving an order. In some embodiments, one or more pharmaceutical medications are repacked after receiving an order.

In some embodiments, the methods further comprise storing a record associated with one or more pharmaceutical medications, e.g., in the memory of an electronic device of the present disclosure. In some embodiments, the record indicates inputted data. In some embodiments, the record indicates one or more location identifiers.

In any of the methods described herein, and at any position or order in the processes of the methods described herein, one or more pharmaceutical medications can be reconciled, e.g., one or more times. One or more pieces of information that can be reconciled between a location and a stored record can include, without limitation, inputted data. For the example, the methods can comprise locating a quantity of one or more pharmaceutical medications at a location in the inventory (e.g., based on a location identifier indicated in a stored record) and reconciling the stored record with the quantity of the one or more pharmaceutical medications located at the location in the inventory (e.g., based on the location identifier indicated in the record). In some embodiments, a user could look up a location identifier at the device (e.g., by accessing one or more stored records), then examine the location based on the location identifier to reconcile its contents as compared to the database. Alternatively, a user could examine a location in the inventory first, then look up a location identifier at the device (e.g., by accessing one or more stored records) to reconcile the location's contents as compared to the database. In some embodiments, reconciling comprises updating a stored record or creating a new record (e.g., in the memory of an electronic device of the present disclosure). For example, a record can be updated or created to reflect a status and/or quantity of a medication at a location in the inventory (or in the entire inventory). In some embodiments, reconciling comprises adjusting or deleting, inputted data of a medication stored at a location in the inventory (or in the entire inventory). In some embodiments, reconciling comprises adjusting or dispositioning, a medication stored at a location in the inventory (or in the entire inventory) in order to match the stored record.

In some embodiments, the methods further comprise receiving (e.g., from an electronic device of the present disclosure) a list of pharmaceutical medication(s) in the inventory that is sorted, grouped, or filtered based at least in part on one or more criteria, information, or inputted data of the present disclosure. This provides the user with a way to view medications of interest, e.g., having a certain name (e.g., brand, trade, generic/non-proprietary, and/or dosage), quantity, value, expiration date, route of administration, therapeutic mechanism or target, indication, manufacturer, labeler, lot or batch number, medication identification code such as an NDC, and the like. This allows for a searchable inventory.

In some embodiments, the methods further comprise updating a record related to one or more pharmaceutical medications to indicate a stored, retrieved, or pending status. For example, while medication(s) are being, or are planned on being, retrieved, dispensed, dispositioned, or otherwise added to or removed from the inventory, the record can indicate a pending or retrieved status. Pending or retrieved status can be used to indicate, e.g., one or more medications that have been, will be, or planned on being ordered, dispensed, recalled, expired, discontinued, and so forth. In some embodiments, this will temporarily removes the medication(s) from searchable inventory, thus preventing access to medication(s) on record that may no longer be stored. In some embodiments, an absent or default status may be used to indicate a status. In some embodiments, a record can be updated to indicate pending or retrieved status prior to retrieving the inventory. In some embodiments, a record can be updated to indicate pending or retrieved status after retrieving the order. In some embodiments, e.g., if the medication(s) are not removed or retrieved from inventory, a record can be updated to change the pending or retrieved status to a stored status. In some embodiments, a record can be updated to change one status to another status automatically, e.g., by an electronic device of the present disclosure. In some embodiments, the record is updated automatically in response to receiving external data (e.g., at the electronic device). For example, the device can receive data representing a tracking number (indicating that one or more medications have been shipped or picked up for shipping) and change a stored or pending status to a retrieved status automatically, e.g., in response to the received data. In some embodiments, the record is updated automatically in response to receiving data inputted at the electronic device. For example, the device can receive inputted data such as a new order and change one status to another automatically, e.g., in response to the inputted data such as a new order, prescription, or refill the status is changed from stored to pending or retrieved. In some embodiments, the record is updated manually at an electronic device (e.g., by a user). In some embodiments, adding or removing one or more statuses may be used to indicate a change in status (e.g. adding a pending indication to, and removing a stored indication from a record would denote a status change from stored to pending).

In some embodiments, the methods further comprise updating a record related to one or more pharmaceutical medications to reflect a quantity (e.g., on-hand) of the medication(s). For example, the record can be updated to subtract a quantity pending or retrieved (e.g., from one or more locations, or in the entire inventory). In some embodiments, the record can be updated to mark at least a portion of the pharmaceutical medication as pending or retrieved. In some embodiments, a more specific marking can indicate a pending or retrieved status in the record. In some embodiments, this more specific marking may indicate that at least a portion of the pharmaceutical medication is one or more of: expired, discontinued, returned, reconciled, missing, inputted in error, repacked, relocated, dispensed, dispositioned, adulterated, misbranded, diverted, expired, recalled, suspect, illegitimate, quarantined, unsafe, out-of-stock, distributed and so forth. In some embodiments, the record is updated after receipt. In some embodiments, the record is updated concurrently with or after storing and/or reconciling. In some embodiments, the record is updated prior to or concurrently with retrieval and/or dispensing or dispositioning. In some embodiments, the record is updated after retrieval and/or dispensing or dispositioning.

In some embodiments of any of the methods described herein, a portion of one or more pharmaceutical medications is dispensed, repacked, or dispositioned, and a second portion (e.g., a remainder) of the one or more pharmaceutical medications is repacked or returned to the inventory. In some embodiments, the second portion is returned to the inventory in a new location. In some embodiments, the second portion is returned to the inventory in the same location (e.g., from whence the first portion was retrieved). In some embodiments, e.g., if the second portion is returned to the inventory in the same location, a record associated with the one or more pharmaceutical medications is updated (e.g., to reflect a new quantity stored at the location). In some embodiments, e.g., if the second portion is returned to the inventory in a new location, a record associated with the one or more pharmaceutical medications is created (e.g., to reflect a quantity stored at the new location).

In some embodiments, one or more of the steps or processes in the methods for dispensing a pharmaceutical medication in an inventory of the present disclosure may be combined, in any number, order, or combination, with one or more of the steps or processes in the methods for storing and/or dispositioning a pharmaceutical medication of the present disclosure.

In some embodiments of any of the methods described herein, and at any position or order in the processes of the methods described herein, data can be received (e.g., from an electronic device of the present disclosure) that is indicative of one or more of: a total quantity of the pharmaceutical medication in the inventory (e.g., stored currently, or stored as of a past or projected date), expiration date(s) of the pharmaceutical medication stored in the inventory, a total quantity of the pharmaceutical medication that has been dispensed, a total quantity of the pharmaceutical medication that has been dispositioned (e.g., disposed, expired, recalled, returned, quarantined), and a total quantity of the pharmaceutical medication that has been reconciled. This allows the user to access information of interest. In some embodiments, the data can be provided to the public, e.g., at a website, application, or web server. In some embodiments, data relating to a future time can be provided, e.g., a month or two in advance (to limit orders received for soon-to-expire medication(s)). In some embodiments, the data can be provided to an auditor or a regulator (e.g., Board of Pharmacy, Department of Public Health, Food and Drug Administration, Drug Enforcement Agency). These features also allow for convenient methods to generate reports based at least in part on one or more criteria.

In some embodiments, after receiving data identifying total quantity of one or more pharmaceutical medications, any of the methods described herein can further comprise, and at any position or order in the processes of the methods described herein: receiving (e.g., from an electronic device of the present disclosure) data identifying a total quantity of a second pharmaceutical medication in the inventory, and based on the received data, displaying total quantities of two or more pharmaceutical medications in the inventory. In some embodiments, the total quantities can be grouped by inputted data. For example, the total quantity may be grouped by one or more of: name, medication identification code such as an NDC, therapeutic area, price, value, status (stored, retrieved, pending, etc.), stock level (high, low, in-stock, out-of-stock), date, user, source, and indication. This allows the user to access and group, search, filter, or sort information of interest related to multiple medications. In some embodiments, the data can be provided to the public, e.g., at a website, application, or web server. For example, received data can identify (for one or more pharmaceutical medications) whether a medication is in inventory (e.g., at a location), not in inventory (e.g., at a location), or pending, and optionally include further information (e.g., a reason why the medication is not in inventory, e.g., at a location).

In some embodiments, one or more quantities displayed are based at least in part on the stored, retrieved, or pending status of at least a portion of the quantity of one or more pharmaceutical medications. In some embodiments, one or more quantities displayed are based at least in part on the expiration date of at least a portion of the quantity of one or more pharmaceutical medications (e.g., displaying only unexpired medications). In some embodiments, one or more quantities displayed are grouped, searchable, filterable, or sortable by inputted data. For example, the quantities displayed can be grouped, searchable, filterable, and/or sortable by one or more of: name, medication identification code such as an NDC, therapeutic area, price, value, status (stored, retrieved, pending, etc.), stock level (high, low, in-stock, out-of-stock), date, user, source, and indication.

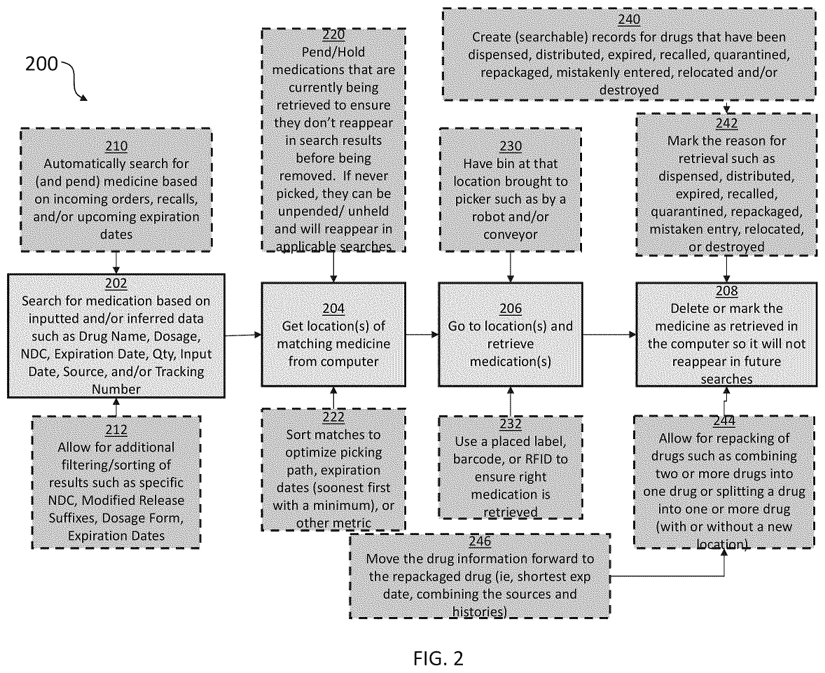

FIG. 2 provides a flowchart for exemplary process 200 that may be used in the methods for dispensing and/or dispositioning a pharmaceutical medication in an inventory of the present disclosure.

At block 202, a search of live inventory (e.g., at an electronic device) is performed by inputting data identifying name, dosage, medication identification code such as an NDC, expiration date, quantity, input date, source, and/or tracking information related to pharmaceutical medication(s). Optionally, at block 210, the data are inputted automatically based on, e.g., an incoming order, recall, or upcoming or past expiration date. Optionally, at block 212, data can be searched, filtered, grouped, or sorted to allow the user to see medication(s) related to a specific medication identification code such as an NDC, modified release suffix, dosage form, or expiration date.

At block 204, location identifier(s) for the pharmaceutical medication(s) is received, e.g., from an electronic device. Optionally, at block 220, medication(s) currently being retrieved are marked as pending. This ensures they will not reappear in a search. They can ultimately be marked as un-pending if stored, and will reappear in future searches. Optionally, at block 222, location identifiers are sorted to optimize suggested path, expiration date (e.g., soonest expiry first, optionally with a minimum date), and the like.

At block 206, a user retrieves the medication(s) using the location identifier(s). Optionally, at block 230, the medication(s) are moved to the user with a robot and/or conveyor. Optionally, at block 232, a label, barcode, or RFID is scanned to ensure correct medication(s).

At block 208, record(s) related to the medication(s) is/are updated or created to indicate pending or retrieved status, and optionally a quantity is identified or updated. This enables a live inventory. Optionally, at block 240, a searchable record is updated or created, e.g., for medication(s) that have been dispensed or dispositioned (e.g. expired, discontinued, returned, reconciled, missing, inputted in error, repacked, relocated, dispensed, dispositioned, adulterated, misbranded, diverted, recalled, suspect, illegitimate, quarantined, unsafe, out-of-stock, and/or distributed). Optionally, at block 242, a reason for retrieval can be stored in a record, (e.g., expired, discontinued, returned, reconciled, missing, inputted in error, repacked, relocated, dispensed, dispositioned, adulterated, misbranded, diverted, recalled, suspect, illegitimate, quarantined, unsafe, out-of-stock, or distributed). Optionally, at block 244, medication(s) are repacked, e.g., by combining two or more drugs into one drug or splitting a drug into two or more drugs (with or without a new location). Optionally, at block 246, information is moved forward to a repackaged medication. For example, the data for the repacked drug can include, without limitation, the original drug name, the original drug location, the shortest expiration date of all the drugs repacked, the total quantity of all the drugs repacked, a list of all sources and transaction information and histories of all the drugs repacked, and references to the records of all drugs repacked. In some embodiments, such reference(s) may be able to trace back all the way to the original manufacturer of the drug, providing a partial or complete record or history of the locations, sources, transactions, and statuses that the drug has encountered. In some embodiments, some or all of this information may be included as a default which can be overwritten by the user.

### III. Methods for Dispositioning a Pharmaceutical Medication

Certain aspects of the present disclosure relate to methods for dispositioning a pharmaceutical medication. Pharmaceutical medication(s) for dispositioning can include, e.g., expired, deleted, discontinued, returned, reconciled, missing, inputted in error, repacked, relocated, dispensed, dispositioned, adulterated, misbranded, diverted, recalled, suspect, illegitimate, quarantined, or unsafe.

In some embodiments, the methods comprise (a) at an electronic device with a processor and memory, inputting data identifying one or more of: an expiration date for one or more pharmaceutical medications and a name for one or more pharmaceutical medications (including, e.g., a medication identification code such as NDC); (b) receiving from the electronic device a location identifier for at least a portion of the one or more pharmaceutical medications, wherein the location identifier identifies a location of at least a portion of the one or more pharmaceutical medications in an inventory, and wherein the ordering of pharmaceutical medications in the inventory is not alphabetical by pharmaceutical medication name; (c) retrieving at least a portion of the one or more pharmaceutical medications from the inventory based on the location identifier; and (d) dispositioning at least a portion of the one or more pharmaceutical medications retrieved in (c).

As used herein, an expired pharmaceutical medication can refer to a medication with a past expiration date, or a medication with an expiration date upcoming within a predetermined amount of time (e.g., less than a typical prescription, based on quantity, frequency of administration, and the expiration date).

As used herein, dispositioning a medication can include one or more of: deleting a record of at least a portion of a pharmaceutical medication from an inventory, updating a record of at least a portion of a pharmaceutical medication as not in an inventory (e.g., not having a “stored” status), quarantining at least a portion of a pharmaceutical medication, disposing at least a portion of a pharmaceutical medication directly or through one or more intermediaries and/or third-parties (e.g., through a reverse distributor, medical waste hauler, or incinerator), and returning at least a portion of a pharmaceutical medication directly or through one or more intermediaries and/or third-parties (e.g., to the originating source such as a wholesaler, labeler, or manufacturer).

In some embodiments, inputted data identify one or more of the manufacturer, labeler, medication identification code such as an NDC, or lot or batch number for one or more recalled pharmaceutical medications. In some embodiments, inputted data further identifies several lot or batch numbers. In some embodiments, inputted data further identifies all lot or batch numbers. For example, a recall can indicate that one or more lots or batches are recalled, that one or more lots or batches are not recalled, or that all lots or batches are recalled.

In some embodiments, the inputted data include a location identifier. In some embodiments, the methods further comprise receiving from the electronic device a record associated with at least a portion of the one or more pharmaceutical medications in the inventory at the location. In some embodiments, the methods further comprise receiving from the electronic device a plurality of records, each record associated with at least a portion of one or more pharmaceutical medications in the inventory (e.g., at a location in the inventory). In some embodiments, the methods further comprise searching, filtering, grouping, or sorting the records of the plurality by one or more of: an expiration date for one or more pharmaceutical medications, a medication identification code or NDC for one or more pharmaceutical medications, and a name for one or more pharmaceutical medications. For example, a user can input a location in the inventory (e.g., by location identifier) and search for record(s) associated with the location, which are sortable by characteristics such as expiration date, medication identification code (e.g., NDC), and name. Advantageously, this can be used to remove certain medication(s) from the location, such as searching for and removing all expired or soon-to-be expired drugs from a location or bin.

In some embodiments, the one or more pharmaceutical medications are non-prescription or over-the-counter medications. In some embodiments, the one or more pharmaceutical medications are prescription medications.

In some embodiments, the location for the pharmaceutical medication in the inventory received from an electronic device of the present disclosure is based on one or more criteria. In some embodiments, the criteria include, in any combination or weighting schema, the inputted data.

In some embodiments, a received order is for two or more pharmaceutical medications in the inventory (e.g., two or more, three or more, four or more, five or more, six or more, seven or more, eight or more, nine or more, ten or more, and so forth). For example, the order (or a second received order) can include information related to a second pharmaceutical medication (e.g., identifying or indicating the second medication's name, quantity, expiration date, and/or medication identification code such as an NDC). In some embodiments, the received order is for one pharmaceutical medication, and fulfillment of the order requires retrieving the pharmaceutical medication from two or more locations in the inventory. For example, an order could be for a quantity of 100, so the user needs to access 4 separate locations, each containing a quantity of 25. In some embodiments, the methods further comprise (e.g., after inputting data related to the one or more pharmaceutical medications at the electronic device) receiving from the electronic device (1) two or more location identifiers of at least a portion of one or more pharmaceutical medications, wherein each of the two or more location identifiers identifies the location of at least a portion of one or more pharmaceutical medications in the inventory, and (2) a suggested path for retrieving at least a portion of the one or more pharmaceutical medications from the inventory (e.g., some or all of the medications in the order(s), including portions thereof) based on the two or more location identifiers; retrieving at least a portion of the one or more pharmaceutical medications from the inventory according to the suggested path; and dispensing or dispositioning the retrieved portion of the one or more pharmaceutical medications. In some embodiments, the suggested path provides a walking path through the inventory for retrieving the medications. In some embodiments, the suggested path provides a suggested order for retrieving the medications in the inventory (e.g., an ordered “pick list”). In some embodiments, the suggested path provides the shortest distance, or an optimized order of retrieval that allows for the shortest distance, to retrieve some or all of the medications from the inventory. In some embodiments, the suggested path identifies two or more locations for the same pharmaceutical medication. Advantageously, this allows the user to retrieve some or all of the medications in an order or orders using an optimized route in the inventory, thereby saving time and labor.

In some embodiments, retrieving the pharmaceutical medication comprises retrieving the whole shelving unit, shelf, inventory container, or section containing the pharmaceutical medication. In some embodiments, one or more pharmaceutical medications (or the shelving unit, shelf, inventory container, or section containing the medication) are retrieved from the inventory manually. For example, a pharmacist or technician can physically enter the inventory and retrieve pharmaceutical medication(s). In some embodiments, one or more pharmaceutical medications (or the shelving unit, shelf, inventory container, or section containing the medication) are retrieved from the inventory using an automated device. For example, the pharmaceutical medication(s) can be moved using a robot, conveyor, or other automated device.

In some embodiments, retrieving one or more pharmaceutical medications from the inventory comprises scanning a code associated with the pharmaceutical medication(s) and/or the location. For example, the code can comprise an optical barcode or RFID code.

In any of the methods described herein, and at any position or order in the processes of the methods described herein, one or more pharmaceutical medications can be repacked, e.g., one or more times. As used herein, repacking refers to modifying the packaging or labeling of one or more pharmaceutical medications, optionally modifying a quantity of the pharmaceutical medication(s) in a single package or container. In some embodiments, repacking comprises combining at least a portion of multiple packages of pharmaceutical medications into one package of pharmaceutical medication. In some embodiments, repacking comprises splitting at least a portion of one package of pharmaceutical medication into multiple packages of pharmaceutical medications. In some embodiments, repacking comprises combining or compounding multiple pharmaceutical ingredients or precursors into a pharmaceutical medication. For example, one or more pharmaceutical medications can be transferred from a blister pack or stock bottle to an amber vial (e.g., for dispensing to a patient) or shipping container (e.g., for dispensing to an intermediary or third party). In another example, upon receipt, pharmaceutical medication(s) can be repacked into a container for holding a different quantity (e.g., transferring from one or more blister packs into a vial or other container with a predetermined quantity, such as a count of 30 or other quantity of the medication that can be typically dispensed as part of a prescription). In some embodiments, one or more pharmaceutical medications are repacked after receipt, but before storing. In some embodiments, one or more pharmaceutical medications are repacked after retrieval, but before dispensing or dispositioning. In some embodiments, the repacking is done by a third party such as by a repackager or relabeler. In some embodiments, a second portion of one or more pharmaceutical medications are repacked upon or after a first portion is dispensed or dispositioned. In some embodiments, one or more pharmaceutical medications are repacked prior to receiving an order. In some embodiments, one or more pharmaceutical medications are repacked after receiving an order.

In some embodiments, the methods further comprise storing a record associated with one or more pharmaceutical medications, e.g., in the memory of an electronic device of the present disclosure. In some embodiments, the record indicates inputted data. In some embodiments, the record indicates one or more location identifiers.

In any of the methods described herein, and at any position or order in the processes of the methods described herein, one or more pharmaceutical medications can be reconciled, e.g., one or more times. One or more pieces of information that can be reconciled between a location and a stored record can include, without limitation, inputted data. For the example, the methods can comprise locating a quantity of one or more pharmaceutical medications at a location in the inventory (e.g., based on a location identifier indicated in a stored record) and reconciling the stored record with the quantity of the one or more pharmaceutical medications located at the location in the inventory (e.g., based on the location identifier indicated in the record). In some embodiments, reconciling comprises updating a stored record or creating a new record (e.g., in the memory of an electronic device of the present disclosure). For example, a record can be updated or created to reflect a status and/or quantity of a medication at a location in the inventory (or in the entire inventory). In some embodiments, reconciling comprises adjusting or deleting, inputted data of a medication stored at a location in the inventory (or in the entire inventory). In some embodiments, reconciling comprises adjusting or dispositioning a medication stored at a location in the inventory (or in the entire inventory) in order to match the stored record.

In some embodiments, the methods further comprise receiving (e.g., from an electronic device of the present disclosure) a list of pharmaceutical medication(s) in the inventory that is sorted, grouped, or filtered based at least in part on one or more criteria, information, or inputted data of the present disclosure. This provides the user with a way to view medications of interest, e.g., having a certain name (e.g., brand, trade, generic/non-proprietary, and/or dosage), quantity, value, expiration date, route of administration, therapeutic mechanism or target, indication, manufacturer, labeler, lot or batch number, medication identification code such as an NDC, and the like. This allows for a searchable inventory.

In some embodiments, the methods further comprise updating a record related to one or more pharmaceutical medications to indicate a stored, retrieved, or pending status. For example, while medication(s) are being, or are planned on being, retrieved, dispensed, dispositioned, or otherwise added to or removed from the inventory, the record can indicate a pending or retrieved status. Pending or retrieved status can be used to indicate, e.g., one or more medications that have been, will be, or planned on being ordered, dispensed, recalled, expired, discontinued, and so forth. In some embodiments, this will temporarily remove the medication(s) from searchable inventory, thus preventing access to medication(s) on record that may no longer be stored. In some embodiments, an absent or default status may be used to indicate a status. In some embodiments, a record can be updated to indicate pending or retrieved status prior to retrieving the inventory. In some embodiments, a record can be updated to indicate pending or retrieved status after retrieving the order. In some embodiments, e.g., if the medication(s) are not removed or retrieved from inventory, a record can be updated to change the pending or retrieved status to a stored status. In some embodiments, a record can be updated to change one status to another status automatically, e.g., by an electronic device of the present disclosure. In some embodiments, the record is updated automatically in response to receiving external data (e.g., at the electronic device). For example, the device can receive data representing a tracking number (indicating that one or more medications have been shipped or picked up for shipping) and change a stored or pending status to a retrieved status automatically, e.g., in response to the received data. In some embodiments, the record is updated automatically in response to receiving data inputted at the electronic device. For example, the device can receive inputted data such as a new order and change one status to another automatically, e.g., in response to the inputted data (e.g., an expiration date or information for a recall or discontinued medications) the status may change from stored to pending or retrieved. In some embodiments, the record is updated manually at an electronic device (e.g., by a user). In some embodiments, adding or removing one or more statuses may be used to indicate a change in status (e.g. adding a pending indication to, and removing a stored indication from a record would denote a status change from stored to pending).

In some embodiments, the methods further comprise updating a record related to one or more pharmaceutical medications to reflect a quantity (e.g., on-hand) of the medication(s). For example, the record can be updated to subtract a quantity pending or retrieved (e.g., from one or more locations, or in the entire inventory). In some embodiments, the record can be updated to mark at least a portion of the pharmaceutical medication as pending or retrieved. In some embodiments, a more specific marking can indicate a pending or retrieved status in the record. In some embodiments, this more specific marking may indicate that at least a portion of the pharmaceutical medication is one or more of: expired, discontinued, returned, reconciled, missing, inputted in error, repacked, relocated, dispensed, dispositioned, adulterated, misbranded, diverted, expired, recalled, suspect, illegitimate, quarantined, unsafe, out-of-stock, distributed and so forth. In some embodiments, the record is updated after receipt. In some embodiments, the record is updated concurrently with or after storing and/or reconciling. In some embodiments, the record is updated prior to or concurrently with retrieval and/or dispensing or dispositioning. In some embodiments, the record is updated after retrieval and/or dispensing or dispositioning.

In some embodiments of any of the methods described herein, a portion of one or more pharmaceutical medications is dispensed, repacked, or dispositioned, and a second portion (e.g., a remainder) of the one or more pharmaceutical medications is repacked or returned to the inventory. In some embodiments, the second portion is returned to the inventory in a new location. In some embodiments, the second portion is returned to the inventory in the same location (e.g., from whence the first portion was retrieved). In some embodiments, e.g., if the second portion is returned to the inventory in the same location, a record associated with the one or more pharmaceutical medications is updated (e.g., to reflect a new quantity stored at the location). In some embodiments, e.g., if the second portion is returned to the inventory in a new location, a record associated with the one or more pharmaceutical medications is created (e.g., to reflect a quantity stored at the new location).

In some embodiments, one or more of the steps or processes in the methods for dispensing a pharmaceutical medication in an inventory of the present disclosure may be combined, in any number, order, or combination, with one or more of the steps or processes in the methods for storing and/or dispositioning a pharmaceutical medication of the present disclosure.

In some embodiments of any of the methods described herein, and at any position or order in the processes of the methods described herein, data can be received (e.g., from an electronic device of the present disclosure) that is indicative of one or more of: a total quantity of the pharmaceutical medication in the inventory (e.g., stored currently, or stored as of a past or projected date), expiration date(s) of the pharmaceutical medication stored in the inventory, a total quantity of the pharmaceutical medication that has been dispensed, a total quantity of the pharmaceutical medication that has been dispositioned (e.g., disposed, expired, recalled, returned, quarantined), and a total quantity of the pharmaceutical medication that has been reconciled. This allows the user to access information of interest. In some embodiments, the data can be provided to the public, e.g., at a website, application, or web server. In some embodiments, data relating to a future time can be provided, e.g., a month or two in advance (to disposition soon-to-expire medication(s)). In some embodiments, the data can be provided to an auditor or a regulator (e.g., Board of Pharmacy, Department of Public Health, Food and Drug Administration, Drug Enforcement Agency). These features also allow for convenient methods to generate reports based at least in part on one or more criteria.

In some embodiments, after receiving data identifying total quantity of one or more pharmaceutical medications, any of the methods described herein can further comprise, and at any position or order in the processes of the methods described herein: receiving (e.g., from an electronic device of the present disclosure) data identifying a total quantity of a second pharmaceutical medication in the inventory, and based on the received data, displaying total quantities of two or more pharmaceutical medications in the inventory. In some embodiments, the total quantities can be grouped by inputted data. For example, the total quantity may be grouped by one or more of: name, medication identification code such as an NDC, therapeutic area, price, value, status (stored, retrieved, pending), stock level (high, low, in-stock, out-of-stock), date, user, source, and indication. This allows the user to access and group, search, filter, or sort information of interest related to multiple medications. In some embodiments, the data can be provided to the public, e.g., at a website, application, or web server. For example, received data can identify (for one or more pharmaceutical medications) whether a medication is in inventory (e.g., at a location), not in inventory (e.g., at a location), or pending, and optionally include further information (e.g., a reason why the medication is not in inventory, e.g., at a location).

In some embodiments, one or more quantities displayed are based at least in part on the stored, retrieved, or pending status of at least a portion of the quantity of one or more pharmaceutical medications. In some embodiments, one or more quantities displayed are based at least in part on the expiration date of at least a portion of the quantity of one or more pharmaceutical medications (e.g., displaying only unexpired medications). In some embodiments, one or more quantities displayed are grouped, searchable, filterable, or sortable by inputted data. For example, the quantities displayed can be grouped, searchable, filterable, and/or sortable by one or more of: name, medication identification code such as an NDC, therapeutic area, price, value, status (stored, retrieved, pending), stock level (high, low, in-stock, out-of-stock), date, user, source, and indication.

FIG. 2 provides a flowchart for exemplary process 200 that may be used in the methods for dispensing and/or dispositioning a pharmaceutical medication in an inventory of the present disclosure.

At block 202, a search of live inventory (e.g., at an electronic device) is performed by inputting data identifying name, dosage, medication identification code such as an NDC, expiration date, quantity, input date, source, and/or tracking information related to pharmaceutical medication(s). Optionally, at block 210, the data are inputted automatically based on, e.g., an incoming order, recall, or upcoming or past expiration date. Optionally, at block 212, data can be searched, filtered, grouped, or sorted to allow the user to see medication(s) related to a specific medication identification code such as an NDC, modified release suffix, dosage form, or expiration date.

At block 204, location identifier(s) for the pharmaceutical medication(s) is received, e.g., from an electronic device. Optionally, at block 220, medication(s) currently being retrieved are marked as pending. This ensures they will not reappear in a search. They can ultimately be marked as un-pending if stored, and will reappear in future searches. Optionally, at block 222, location identifiers are sorted to optimize suggested path, expiration date (e.g., soonest expiry first, optionally with a minimum date), and the like.

At block 206, a user retrieves the medication(s) using the location identifier(s). Optionally, at block 230, the medication(s) are moved to the user with a robot and/or conveyor. Optionally, at block 232, a label, barcode, or RFID is scanned to ensure correct medication(s).

At block 208, record(s) related to the medication(s) is/are updated or created to indicate pending or retrieved status, and optionally a quantity is identified or updated. This enables a live inventory. Optionally, at block 240, a searchable record is updated or created, e.g., for medication(s) that have been dispensed or dispositioned (e.g. expired, discontinued, returned, reconciled, missing, inputted in error, repacked, relocated, dispensed, dispositioned, adulterated, misbranded, diverted, recalled, suspect, illegitimate, quarantined, unsafe, out-of-stock, and/or distributed). Optionally, at block 242, a reason for retrieval can be stored in a record, (e.g., expired, discontinued, returned, reconciled, missing, inputted in error, repacked, relocated, dispensed, dispositioned, adulterated, misbranded, diverted, recalled, suspect, illegitimate, quarantined, unsafe, out-of-stock, or distributed). Optionally, at block 244, medication(s) are repacked, e.g., by combining two or more drugs into one drug or splitting a drug into two or more drugs (with or without a new location). Optionally, at block 246, information is moved forward to a repackaged medication. For example, the data for the repacked drug can include, without limitation, the original drug name, the original drug location, the shortest expiration date of all the drugs repacked, the total quantity of all the drugs repacked, a list of all sources and transaction information and histories of all the drugs repacked, and references to the records of all drugs repacked. In some embodiments, such references may be able to traced back all the way to the original manufacturer of the drug, providing a partial or complete record or history of the locations, sources, transactions, and statuses that the drug has encountered. In some embodiments, some or all of this information may be included as a default which can be overwritten by the user.

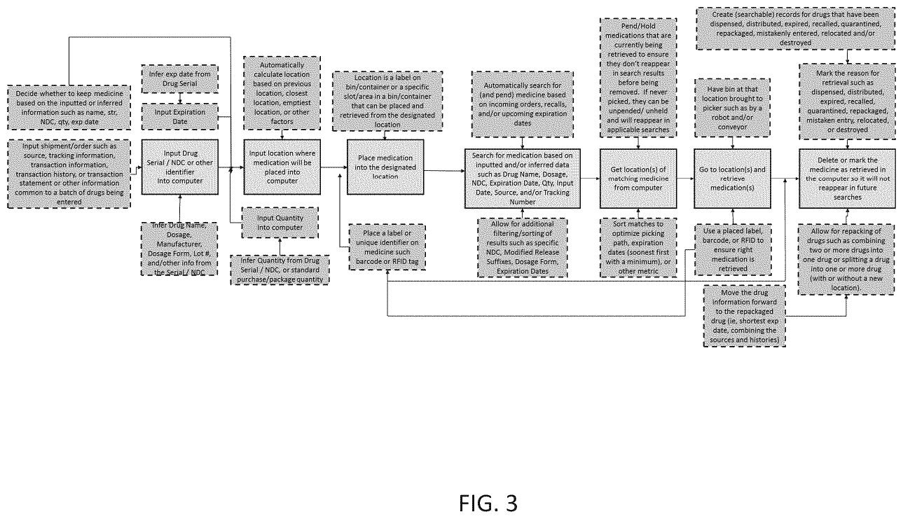

FIG. 3 provides a flowchart for how methods of storing, dispensing, and dispositioning can be combined, e.g., by integrating exemplary processes 100 and 200. For example, the location inputted at block 104 and used for storage at block 106 can be the location received at block 204 and from which the received and/or stored medication can be retrieved at block 206. As another non-limiting example, the label associated at block 132 can be scanned to ensure correct retrieval at block 232.

### IV. Electronic Devices

Certain aspects of the present disclosure relate to electronic devices, e.g., for use in the methods described supra. In some implementations, the device may include a processor and memory. As used herein, a processor may include any processor known in the art suitable for a computer-implemented system described herein. As used herein, memory may include high-speed random access memory and/or non-volatile memory, or any other suitable memory known in the art. In some implementations, the memory may be configured to include computer readable instructions for receiving recipient and donor information, as described herein. In some implementations, the memory may be configured to include computer readable instructions for receiving inputted data, storing records, providing a location identifier, and the like, as described above.

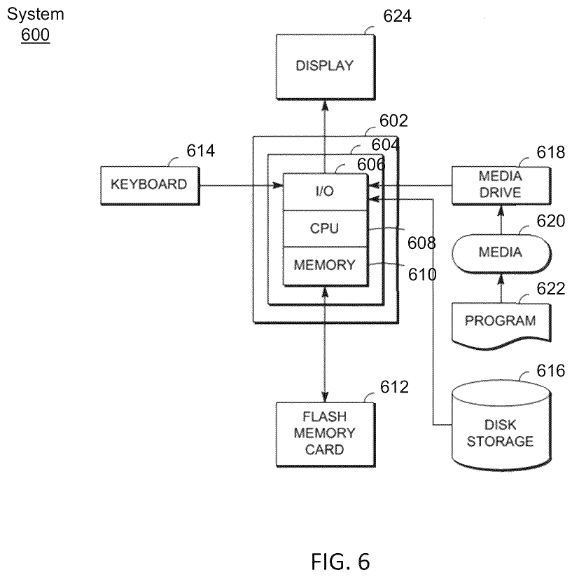

FIG. 6 depicts an exemplary electronic device, computing system 600, with a number of components that can be used to perform the above-described processes. The main system 602 includes a motherboard 604 having an I/O section 606, one or more central processing units (CPU) 608, and a memory section 610, which may have a flash memory card 612 related to it. The I/O section 606 is connected to a display 624, a keyboard 614, a disk storage unit 616, and a media drive unit 618. The media drive unit 618 can read/write a computer-readable medium 620, which can contain programs 622 and/or data.

At least some values based on the results of the above-described processes can be saved for subsequent use. Additionally, a computer-readable medium can be used to store (e.g., tangibly embody) one or more computer programs for performing any one of the above-described processes by means of a computer. The computer program may be written, for example, in a general-purpose programming language (e.g., Javascript, Python, Go, Rust, Pascal, C, C++, Java) or some specialized application-specific language.

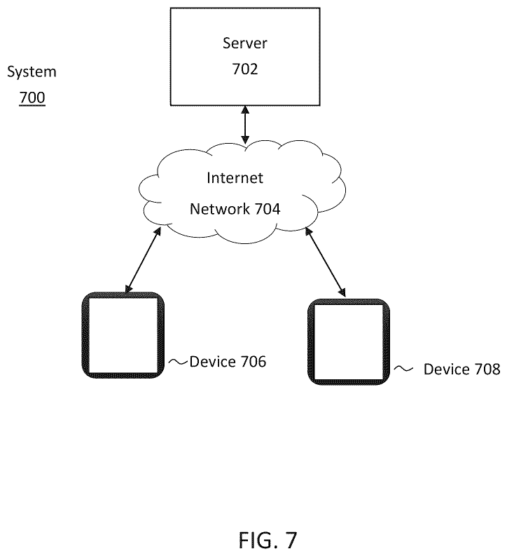

FIG. 7 depicts server 702 and client devices 706 and 708 (e.g., devices that may be used in a pharmacy by a pharmacist or technician) may include any one of various types of computer devices, having, e.g., a processing unit, a memory (which may include logic or software for carrying out some or all of the functions described herein), and a communication interface, as well as other conventional computer components (e.g., input device, such as a keyboard/touch screen, and output device, such as display). For example, client devices 706 and 708 may include a desktop computer, mobile device such as a mobile phone, web-enabled phone, or smart phone.

Devices 706/708 and server 702 may communicate, e.g., using suitable communication interfaces via a network 704, such as a Local Area Network (LAN) or the Internet. Devices 706/708 and server 702 may communicate, in part or in whole, via wireless or hardwired communications, such as Ethernet, IEEE 802.11b wireless, or the like. Additionally, devices 706/708 and server 702 may communicate, e.g., using suitable communication interfaces, via a second network, such as a mobile/cellular network. Communication between devices 706/708 and server 702 may further include or communicate with various servers such as a mail server, mobile server, media server, telephone server, and the like.

One or all of devices 706/708 and server 702 generally include logic (e.g., http web server logic) or is programmed to format data, accessed from local or remote databases or other sources of data and content, for providing and/or receiving information via network 704 according to various examples described herein. To this end, server 702 may utilize various web data interface techniques such as Common Gateway Interface (CGI) protocol and associated applications (or “scripts”), Java® “servlets”, i.e., Java® applications running on server 702, or the like to present information and receive input from devices 706/708. The server 702, although described herein in the singular, may actually comprise plural computers, devices, databases, associated backend devices, and the like, communicating (wired and/or wireless) and cooperating to perform some or all of the functions described herein. Server 702 may further include or communicate with account servers (e.g., email servers), mobile servers, media servers, and the like.

It should be noted that although the exemplary methods and systems described herein describe use of a separate server and database systems for performing various functions, other embodiments could be implemented by storing the software or programming that operates to cause the described functions on a single device or any combination of multiple devices as a matter of design choice so long as the functionality described is performed. Similarly, the database system described can be implemented as a single database, a distributed database, a collection of distributed databases, a database with redundant online or offline backups or other redundancies, or the like, and can include a distributed database or storage network and associated processing intelligence. Although not depicted in the figures, server 702 (and other servers and services described herein) generally include such art recognized components as are ordinarily found in server systems, including but not limited to processors, RAM, ROM, clocks, hardware drivers, associated storage, and the like. Further, the described functions and logic may be included in software, hardware, firmware, or combination thereof.

Exemplary embodiments of the present disclosure are provided below. These embodiments illustrate exemplary aspects of the present disclosure and are not intended to limit the present disclosure.

Embodiment 1: A method for storing at least a portion of a pharmaceutical medication in an inventory, comprising:
(a) receiving a pharmaceutical medication;
(b) at an electronic device with a processor and memory, inputting data related to the pharmaceutical medication, wherein the data identify:
   (1) name and quantity of at least a portion of the pharmaceutical medication, and
   (2) a location for at least a portion of the pharmaceutical medication in an inventory, wherein the ordering of pharmaceutical medications in the inventory is not alphabetical by pharmaceutical medication name;
(c) creating a record associated with at least a portion of the pharmaceutical medication in the memory of the electronic device, wherein the record identifies the name and quantity of the portion of the pharmaceutical medication and the location for the portion of the pharmaceutical medication in the inventory; and
(d) storing the portion of the pharmaceutical medication at the location in the inventory.

Embodiment 2: The method of embodiment 1, wherein the data inputted in (b) are derived at least in part from documentation associated with the pharmaceutical medication(s) received in (a).

Embodiment 3: The method of embodiment 1 or embodiment 2, wherein the first pharmaceutical medication is received in a first shipment in (a), and the method further comprises:
after (d), receiving a second shipment comprising the pharmaceutical medication;
at the electronic device, inputting second data related to the pharmaceutical medication, wherein the second data identify:
   name of the pharmaceutical medication and quantity of at least a portion of the pharmaceutical medication received in the second shipment, and
   a second location for at least a portion of the pharmaceutical medication in the inventory, wherein the second location of the pharmaceutical medication in the inventory is different from the first location of the pharmaceutical medication in the inventory inputted in (b);
creating a second record associated with the pharmaceutical medication in the memory of the electronic device, wherein the second record identifies the second location for the pharmaceutical medication in the inventory and a quantity of the pharmaceutical medication based at least in part on the quantity of the portion of the pharmaceutical medication received in the second shipment; and
storing the portion of the pharmaceutical medication received in the second shipment at the second location in the inventory.

Embodiment 4: The method of embodiment 1 or embodiment 2, wherein the location for the pharmaceutical medication in the inventory inputted in (b) is different from the previous location for a pharmaceutical medication having the same name or medication identification code as the pharmaceutical medication received in (a).

Embodiment 5: The method of any one of embodiments 1-4, further comprising, after (c): updating a record of at least a portion of the pharmaceutical medication in the memory of the electronic device to add the stored portion.

Embodiment 6: The method of any one of embodiments 1-5, further comprising, after (a), associating a code with the pharmaceutical medication.

Embodiment 7: The method of embodiment 6, wherein the code comprises an optical barcode.

Embodiment 8: The method of embodiment 6, wherein the code comprises a radio frequency identification (RFID) code.

Embodiment 9: The method of any one of embodiments 1-8, wherein the data are inputted in (b) through an optical barcode scanner, RFID reader, or OCR device.

Embodiment 10: The method of any one of embodiments 1-9, wherein the location is automatically inputted by the electronic device in (b) based on one or more criteria.

Embodiment 11: The method of embodiment 10, wherein the criteria comprise one or more of: a previous location in the inventory, a proximal location in the inventory, an available location in the inventory, name, expiration date, source, medication identification code, NDC, dosage form, manufacturer, labeler, lot or batch number, quantity, count, cost, price, value, packaging size or type, the inventory turnover, storage requirement, tracking information, shipment method, courier, date created, date updated, date shipped, date received, date stored, date pended, date retrieved, date dispensed, date dispositioned, transaction information or history, and condition of the pharmaceutical medication.

Embodiment 12: The method of any one of embodiments 1-11, further comprising, after (a)-(c), associating an expiry indicator with the pharmaceutical medication.

Embodiment 13: The method of any one of embodiments 1-12, wherein the location identifier for the portion of the pharmaceutical medication in the inventory identifies one or more of: an aisle or area, a shelving unit, an inventory container, and a section within an aisle, area, shelving unit, or inventory container.

Embodiment 14: The method of any one of embodiments 1-13, wherein the inventory comprises a plurality of sections, and wherein each section of the plurality is labeled with a different location in the inventory.

Embodiment 15: The method of embodiment 14, wherein the sections of the plurality are designated or divided by physical dividers that are movable within the inventory.

Embodiment 16: The method of embodiment 14 or embodiment 15, wherein at least a portion of the inventory is at room temperature.

Embodiment 17: The method of any one of embodiments 14-16, wherein at least a portion of the inventory is cooler than room temperature.

Embodiment 18: The method of any one of embodiments 1-17, further comprising, after (d):
(e) receiving an order for at least a portion of a pharmaceutical medication received in (a), wherein the order identifies a name and a quantity for the portion of the pharmaceutical medication;
(f) at the electronic device, inputting data related to at least a portion of the pharmaceutical medication, wherein the data identify the name and quantity for the portion of the pharmaceutical medication;
(g) receiving a location identifier for at least a portion of the pharmaceutical medication from the electronic device, wherein the location identifier identifies the location of the portion of the pharmaceutical medication in the inventory;
(h) retrieving at least a portion of the pharmaceutical medication from the inventory based on the location identifier(s); and
(i) dispensing at least a portion of the pharmaceutical medication retrieved in (h) based on the received order.

Embodiment 19: A method for dispensing a portion of one or more pharmaceutical medications, comprising:
(a) receiving an order for one or more pharmaceutical medications, wherein the order indicates a name and a quantity for each of the one or more pharmaceutical medications;
(b) at an electronic device with a processor and memory, inputting data related to at least a portion of the one or more pharmaceutical medications, wherein the data identify the name and quantity for the portion of the one or more pharmaceutical medications;
(c) receiving a location identifier for at least a portion of the one or more pharmaceutical medications from the electronic device, wherein the location identifier identifies a location of the portion of the one or more pharmaceutical medications in an inventory, and wherein the ordering of pharmaceutical medications in the inventory is not alphabetical by pharmaceutical medication name;
(d) retrieving at least a portion of the one or more pharmaceutical medications from the inventory based on the location identifier(s); and
(e) dispensing at least a portion of the one or more pharmaceutical medications retrieved in (d) based on the received order.

Embodiment 20: The method of embodiment 19, wherein the order comprises a prescription or refill for each of the one or more prescription pharmaceutical medications, and wherein the prescription or refill identifies the name and quantity for the one or more prescription pharmaceutical medications.

Embodiment 21: The method of embodiment 19 or embodiment 20, wherein the order is received automatically at the electronic device in (a), and wherein the data related to at least a portion of the one or more pharmaceutical medications are inputted automatically at the electronic device in (b).

Embodiment 22: The method of any one of embodiments 19-21, wherein the location identifier received in (c) identifies a location of a generic or therapeutic equivalent of at least a portion of the one or more pharmaceutical medications from the order received in (a), and wherein the generic or therapeutic equivalent of at least a portion of the one or more pharmaceutical medications is retrieved in (d) and dispensed in (e).

Embodiment 23: The method of any one of embodiments 19-22, wherein the location identifier received from the electronic device is based on one or more criteria.

Embodiment 24: The method of embodiment 23, wherein the criteria comprise one or more of: a previous location in the inventory, a proximal location in the inventory, an available location in the inventory, name, expiration date, source, medication identification code, NDC, dosage form, manufacturer, labeler, lot or batch number, quantity, count, cost, price, value, packaging size or type, the inventory turnover, storage requirement, tracking information, shipment method, courier, repacked by user, reconciled by user, created by user, inputted by user, updated by user, shipped by user, received by user, stored by user, pended by user, retrieved by user, dispensed by user, dispositioned by user, date repacked, date reconciled, date created, date inputted, date updated, date shipped, date received, date stored, date pended, date retrieved, date dispensed, date dispositioned, transaction information or history, and condition of the pharmaceutical medication.

Embodiment 25: A method for dispositioning at least a portion of one or more pharmaceutical medications, comprising:
(a) at an electronic device with a processor and memory, inputting data identifying one or more of: an expiration date for one or more pharmaceutical medications and a name for one or more pharmaceutical medications;
(b) receiving from the electronic device a location identifier for at least a portion of the one or more pharmaceutical medications, wherein the location identifier identifies a location of at least a portion of the one or more pharmaceutical medications in an inventory, and wherein the ordering of pharmaceutical medications in the inventory is not alphabetical by pharmaceutical medication name;
(c) retrieving at least a portion of the one or more pharmaceutical medications from the inventory based on the location identifier; and
(d) dispositioning at least a portion of the one or more pharmaceutical medications retrieved in (c).

Embodiment 26: The method of embodiment 25, wherein a location identifier is inputted in (a), and wherein (b) further comprises receiving from the electronic device a record, wherein the record is associated with at least a portion of the one or more pharmaceutical medications in the inventory at the location.

Embodiment 27: The method of embodiment 26, wherein (b) further comprises receiving from the electronic device a plurality of records, each record associated with at least a portion of one or more pharmaceutical medications in the inventory, and wherein the method further comprises searching, filtering, grouping, or sorting the records of the plurality by one or more of: an expiration date for one or more pharmaceutical medications, a medication identification code or NDC for one or more pharmaceutical medications, and a name for one or more pharmaceutical medications.

Embodiment 28: The method of any one of embodiments 25-27, wherein the data inputted in (a) identifies the NDC, manufacturer, or labeler for one or more recalled pharmaceutical medications and further identifies a lot or batch number for the one or more recalled pharmaceutical medications.

Embodiment 29: The method of any one of embodiments 1-28, wherein the inputted data further identify one or more of: a previous location in the inventory, a proximal location in the inventory, an available location in the inventory, name, expiration date, source, medication identification code, NDC, dosage form, manufacturer, labeler, lot or batch number, quantity, count, cost, price, value, packaging size or type, the inventory turnover, storage requirement, tracking information, shipment method, courier, repacked by user, reconciled by user, created by user, inputted by user, updated by user, shipped by user, received by user, stored by user, pended by user, retrieved by user, dispensed by user, dispositioned by user, date repacked, date reconciled, date created, date inputted, date updated, date shipped, date received, date stored, date pended, date retrieved, date dispensed, date dispositioned, transaction information or history, and condition of the pharmaceutical medication.

Embodiment 30: The method of any one of embodiments 19-29, wherein a first portion of the one or more pharmaceutical medications is dispensed or dispositioned, and wherein the method further comprises one or both of: repacking and returning to the inventory a second portion of the one or more pharmaceutical medications.

Embodiment 31: The method of embodiment 30, wherein the second portion of the one or more pharmaceutical medications is returned to the inventory in a new location.

Embodiment 32: The method of any one of embodiments 19-31, further comprising:
after inputting the data related to the one or more pharmaceutical medications at the electronic device, receiving from the electronic device:
   (1) two or more location identifiers of at least a portion of one or more pharmaceutical medications, wherein each of the two or more location identifiers identifies the location of at least a portion of one or more pharmaceutical medications in the inventory, and
   (2) a suggested path for retrieving at least a portion of the one or more pharmaceutical medications from the inventory based on the two or more location identifiers;
retrieving at least a portion of the one or more pharmaceutical medications from the inventory according to the suggested path; and
dispensing or dispositioning the retrieved portion of the one or more pharmaceutical medications.

Embodiment 33: The method of any one of embodiments 19-32, wherein retrieving the portion of the pharmaceutical medication from the inventory is performed manually.

Embodiment 34: The method of any one of embodiments 19-32, wherein retrieving the portion of the pharmaceutical medication from the inventory is performed by a robot, conveyor, or other automated device.

Embodiment 35: The method of any one of embodiments 19-34, further comprising, prior to dispensing or dispositioning the one or more pharmaceutical medications: repacking the one or more pharmaceutical medications.

Embodiment 36: The method of any one of embodiments 19-35, further comprising, prior to dispensing or dispositioning the one or more pharmaceutical medications:

reconciling a quantity of the one or more pharmaceutical medications at the location in the inventory based on the one or more location identifiers.

Embodiment 37: The method of any one of embodiments 19-36, further comprising, after inputting the data:
receiving from the electronic device a list of one or more pharmaceutical medications in the inventory; and
one or more of sorting, grouping, and filtering the list based at least in part on the inputted data.

Embodiment 38: The method of any one of embodiments 19-37, further comprising, after inputting the data: updating a record of the portion of the one or more pharmaceutical medications in the memory of the electronic device to indicate pending or received status.

Embodiment 39: The method of any one of embodiments 1-38, further comprising: receiving from the electronic device data identifying total quantity, count, cost, price, or value of at least a portion of the one of the pharmaceutical medications which are stored, pending, or retrieved.

Embodiment 40: The method of any one of embodiments 19-39, further comprising: updating a record of at least a portion of the one or more pharmaceutical medications in the memory of the electronic device to subtract the pending or retrieved portion.

Embodiment 41: The method of any one of embodiments 19-39, further comprising: updating a record of at least a portion of the one or more pharmaceutical medications in the memory of the electronic device to indicate the pending or retrieved portion as pending or retrieved.

Embodiment 42: The method of any one of embodiments 19-39, further comprising: updating a record of at least a portion of the one or more pharmaceutical medications in the memory of the electronic device to indicate a status of the portion of the one or more pharmaceutical medications as available.

Embodiment 43: The method of any one of embodiments 40-42, wherein the record is updated automatically by the electronic device.

Embodiment 44: The method of any one of embodiments 19-43, wherein retrieving the pharmaceutical medication from the inventory in (d) comprises scanning one or more codes associated with one or both of the pharmaceutical medication and the location.

Embodiment 45: The method of embodiment 44, wherein the one or more codes comprise an optical barcode.

Embodiment 46: The method of embodiment 44, wherein the one or more codes comprise a radio frequency identification (RFID) code.

Although only certain exemplary embodiments have been described in detail above, those skilled in the art will readily appreciate that many modifications are possible in the exemplary embodiments without materially departing from the novel teachings and advantages of this disclosure. For example, aspects of embodiments disclosed above can be combined in other combinations to form additional embodiments. Accordingly, all such modifications are intended to be included within the scope of this technology.

The foregoing description, for purpose of explanation, has been described with reference to specific implementations. However, the illustrative discussions above are not intended to be exhaustive or to limit the disclosed implementations to the precise forms disclosed. Many modifications and variations are possible in view of the above teachings. The implementations were chosen and described in order to best explain the principles and practical applications of the disclosed ideas, to thereby enable others skilled in the art to best utilize them with various modifications as are suited to the particular use contemplated.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

1-46. (canceled)

47. A method for storing a pharmaceutical medication in an inventory, comprising:
(a) obtaining, via an optical scanner, image data that is associated with a pharmaceutical medication and corresponds to one or more of the following: medication packaging, order invoice(s), order receipt(s), shipping manifest(s), packing slip(s), tracking code(s), transaction statement(s), transaction information, and transaction history;
(b) at an electronic device with a processor and memory, inputting the image data into an Optical Character Recognition (OCR), Neural Network, Deep Learning, and/or Natural Language Processing (NLP) model to identify a medication identification code of the pharmaceutical medication;
(c) creating, via the processor, a database record associated with the pharmaceutical medication, wherein the database record identifies the medication identification code;
(d) storing the database record in the memory of the electronic device; and
(e) storing the pharmaceutical medication in an inventory.

48. The method of claim 47, further comprising:
inferring one or more of a medication name, quantity, dosage, dosage form, manufacturer, labeler, lot or batch number, or expiration date from the medication identification code via the OCR, Neural Network, Deep Learning, and/or NLP model; and
storing the inferred name, quantity, dosage, dosage form, manufacturer, labeler, lot or batch number, and/or expiration date in the database record.

49. The method of claim 47, wherein the inputted image data further identify one or more of a medication condition, expiration date, dosage form, strength, storage requirement, route of administration, therapeutic mechanism or target, indication, and source pharmacy associated with the pharmaceutical medication.

50. The method of claim 47, further comprising, after (d), reconciling the medication identification code associated with the database record with a medication identification code of the pharmaceutical medication stored in the inventory.

51. The method of claim 47, further comprising, after (d), reconciling one or more of a name, quantity, dosage, manufacturer, labeler, dosage form, lot or batch number, or expiration date associated with the database record with a name, quantity, dosage, manufacturer, labeler, dosage form, lot or batch number, and/or expiration date of the pharmaceutical medication stored in the inventory.

52. The method of claim 47, wherein the pharmaceutical medication is stored in the inventory at a location marked for disposition or quarantine.

53. The method of claim 47, wherein the ordering of pharmaceutical medications in the inventory is not alphabetical by pharmaceutical medication name, and wherein the database record further identifies a location for the pharmaceutical medication in the inventory.

54. The method of claim 49, wherein the database record further identifies one or more of the medication condition, medication dosage form, and medication storage requirement.

55. The method of claim 49, wherein the inputted image data further identify an expiration date associated with the pharmaceutical medication, and the database record further identifies the expiration date.

56. The method of claim 47, further comprising:
(f) receiving an order for the pharmaceutical medication;
(g) receiving from the memory of the electronic device a list comprising a plurality of database records for fulfilling the received order, wherein each database record of the list identifies a name and quantity of a pharmaceutical medication from the inventory, wherein the database records of the list are sorted, grouped, or filtered based at least in part on the received order; and
(h) retrieving, from the memory of the electronic device, one or more database record(s) of the plurality that is/are associated with a pharmaceutical medication from the received order, wherein each of the one or more database record(s) identifies a name, quantity, and location of the pharmaceutical medication in the inventory.

57. The method of claim 56, further comprising retrieving the pharmaceutical medication from the inventory using a robot, conveyor, or other automated device.

58. The method of claim 56, further comprising, after (g), updating one or more database record(s) of the plurality to indicate a stored, retrieved, or pending status.

59. The method of claim 56, wherein the database records of the list are sorted, grouped, or filtered based at least in part on one or more of: name, quantity, value, expiration date, route of administration, therapeutic mechanism or target, indication, manufacturer, labeler, lot or batch number, and medication identification code.

60. The method of claim 56, further comprising retrieving the pharmaceutical medication from the inventory.

61. The method of claim 56, wherein the received order is for at least two pharmaceutical medications, and the method further comprises receiving from the electronic device:
two or more location identifiers, wherein each of the two or more location identifiers identifies a location of one of the pharmaceutical medications in the inventory that can be used to fulfill the received order; and
a suggested path for retrieving the at least two pharmaceutical medications from the corresponding locations in the inventory based on the two or more location identifiers.

62. The method of claim 56, wherein the method further comprises receiving from the electronic device:
two or more location identifiers, wherein each of the two or more location identifiers identifies a different location for a portion of the same pharmaceutical medication from the received order in the inventory; and
a suggested path for retrieving a portion of the pharmaceutical medication from each of the corresponding locations in the inventory based on the two or more location identifiers.

63. The method of claim 47, further comprising, using the processor and memory, inputting data related to the pharmaceutical medication via an optical barcode scanner or radio frequency identification (RFID) reader.

64. The method of claim 47, wherein the medication identification code is identified by the OCR, Neural Network, Deep Learning, and/or NLP model based on image data corresponding to the medication packaging.

65. The method of claim 47, wherein one or more of quantity, expiration date, lot or batch number, manufacturer, labeler, and storage requirement of the pharmaceutical medication is/are identified by the OCR, Neural Network, Deep Learning, and/or NLP model based on image data corresponding to the medication packaging.

66. The method of claim 47, wherein inputting the image data into the OCR, Neural Network, Deep Learning, and/or NLP model comprises processing the image data using one or more centralized or distributed databases, Neural Networks, Deep Learning, and/or Artificial Intelligence (AI) models.

67. A system, comprising:
a memory configured to store one or more program instructions; and
one or more processors configured to execute the one or more program instructions, wherein the one or more program instructions when executed by the one or more processors are configured to:
obtain image data corresponding to one or more of the following associated with a pharmaceutical medication: medication packaging, order invoice(s), order receipt(s), shipping manifest(s), packing slip(s), tracking code(s), transaction statement(s), transaction information, and transaction history;
input the image data into an Optical Character Recognition (OCR), Neural Network, Deep Learning, and/or Natural Language Processing (NLP) model stored in the memory to identify a medication identification code of the pharmaceutical medication;
create, via the one or more processors, a database record associated with the pharmaceutical medication, wherein the database record identifies the medication identification code of the pharmaceutical medication; and
store the database record in the memory.
